(19) United States
(12) Patent Application Publication
(10) Pub. No.: US 2025/0008958 A1
(43) Pub. Date: Jan. 9, 2025

(54) BROAD SPECTRUM ANTI-MICROBIAL POLYMERIC COMPOSITION

(71) Applicant: CORNELL UNIVERSITY, Ithaca, NY (US)

(72) Inventors: Minglin MA, Ithaca, NY (US); Qingsheng LIU, Ithaca, NY (US)

(21) Appl. No.: 18/684,335

(22) PCT Filed: Aug. 17, 2022
(86) PCT No.: PCT/US2022/040545

§ 371 Date: Feb. 16, 2024

Related U.S. Application Data
(60) Provisional application No. 63/234,457, filed on Aug. 18, 2021.

(51) Int. Cl. A01N 43/72; A01P 1/00; A01P 3/00; C08G 71/04

(52) U.S. Cl. CPC A01N 43/72; A01P 1/00; A01P 3/00; C08G 71/04

(57) ABSTRACT

A polymeric composition comprising a polyurethane containing at least the following functional groups: (i) a five-membered or six-membered heterocyclic ring containing at least one ring heteroatom selected from nitrogen and oxygen atoms, and (ii) a zwitterionic group. Also described herein are articles made of the polymeric composition. Also described herein are methods for inactivating microbes and viruses on a surface of an object, the method comprising incorporating the anti-microbial polymeric composition into a surface of the object, either by constructing an object of the polymeric composition or by coating the surface of an object with the polymeric composition.

### CROSS REFERENCE TO RELATED APPLICATIONS

This application claims the benefit of priority from U.S. Provisional Application No. 63/234,457, filed on Aug. 18, 2021, which is herein incorporated by reference in its entirety.

### FIELD OF THE INVENTION

The present invention generally relates to anti-microbial polymeric compositions, and more particularly, such compositions that can be applied as coatings on an object to make the surface of the object anti-microbial.

### BACKGROUND OF THE INVENTION

The COVID-19 pandemic, caused by the severe acute respiratory syndrome coronavirus 2 (SARS-COV-2), has infected millions of people and resulted in a substantial number of deaths worldwide since its outbreak. Studies have shown that SARS-COV-2 remains viable and infectious in aerosols for hours and on surfaces for days, thus posing a risk for further transmission. As a result, in addition to social distancing and mask-wearing, frequent surface decontamination has become an important preventative measures to minimize transmission risks. Liquid-based disinfectants, such as chlorine-based products, have been widely used during this pandemic due to their antiviral potency and history of relatively safe use. However, these disinfectants decontaminate the surface only at the time of application and do not provide long-lasting protection. In other efforts, copper-based surfaces or those coated with polymeric quaternary ammonium compounds have prolonged antiviral properties. However, these surfaces often require drying or long contact time (e.g., hours) with the viral contaminants to be effective.

### SUMMARY OF THE INVENTION

In one aspect, the present disclosure is directed to polymeric compositions having the ability to inactivate microbes and viruses, particularly those microbes that cause infection by contact with a contaminated surface or by spreading through drinking water. The microbe may be, for example, a virus, a bacterium (e.g., E. Coli. Streptococcus, Staphylococcus, Salmonella, Shigella, or Mycobacterium), archaebacterium, protist, or fungus, or a combination of any of these. The virus may be, for example, a coronavirus (e.g., SARS or COVID), influenza, rhinovirus, norovirus, chickenpox, herpes, mononucleosis, viral meningitis, mumps, measles, rubella, viral hepatitis, or human immunodeficiency virus (HIV), or a combination of any of these. The virus may also be an enveloped or non-enveloped virus.

The polymeric composition is or includes a polyurethane composition that contains at least the following functional groups: (i) a five-membered or six-membered heterocyclic ring containing at least one ring heteroatom selected from nitrogen and oxygen atoms, and (ii) a zwitterionic group. In some embodiments, functional group (1) contains precisely or at least one ring nitrogen atom or precisely or at least one ring oxygen atom. In other embodiments, functional group (i) contains at least two ring heteroatoms selected from nitrogen and oxygen heteroatoms, e.g., two or more ring nitrogen atoms, two or more ring oxygen atoms, or at least one ring nitrogen atom and at least one ring oxygen atom.

In some embodiments, functional group (i) contains a halamine, which is a halogen atom (e.g., chlorine, bromine, or iodine atom) coordinated (i.e., bound) to a nitrogen atom in the polymeric composition. The nitrogen atom typically belongs to a primary amine (—NH₂) or secondary amine (e.g., —NH—) before being converted to the halamine (by replacement of the hydrogen atom in the amine with a halogen atom, such as chlorine). In some embodiments, the nitrogen atom coordinated to the halogen atom may be a nitrogen atom in functional group (i), e.g., a ring nitrogen atom or amino-containing group in functional group (i). In other embodiments, the nitrogen atom coordinated to the halogen atom may be located in a urethane linkage, urea linkage, or a pendant amino-containing group in the polyurethane. In an exemplary embodiment, the halamine is a chlorine atom coordinated to a ring nitrogen atom of functional group (i). The chlorine or other halogen atom provides the polymeric composition with additional ability to inactivate a microbe or virus.

Functional group (ii), the zwitterionic group, can be any zwitterionic group known in the art, such as a betaine group, or more specifically, a sulfobetaine, carboxybetaine, or phosphobetaine (phosphonium betaine) group. In particular embodiments, the zwitterionic group is a sulfobetaine group, such as n-tetradecyl-N,N-dimethyl-3-ammonio-1-propanesulfonate, n-octyl-N,N-dimethyl-3-ammonio-1-propanesulfonate, n-decyl-N,N-dimethyl-3-ammonio-1-propanesulfonate, and n-dodecyl-N,N-dimethyl-3-ammonio-1-propanesulfonate.

Moreover, in a first embodiment, the term “polyurethane” refers to first and second polyurethanes in homogeneous admixture (i.e., as a blend), wherein the first polyurethane contains functional group (i) and not functional group (ii), and the second polyurethane contains functional group (ii) and not functional group (i); while, in a second embodiment, the term “polyurethane” refers to a single polyurethane containing functional groups (i) and (ii).

The polymeric composition may also be made to assume any of a variety of shapes, including a film or membrane shape. In the case of a film or membrane, the film or membrane typically functions as a coating on an underlying object for which microbial or viral resistance is desired. In some embodiments, the polymeric composition is porous. In other embodiments, the polymeric composition is non-porous. In some embodiments, the polymeric composition has a fibrous structure, i.e., it is constructed of fibers that are bonded or unbonded to each other, wherein the fibers may be woven or non-woven and may be solid or hollow. The fibers can be produced by any suitable method, such as melt spinning or electrospinning. In various embodiments, the fibers may have a diameter within a range of 0.1-1000 microns, 0.1-500 microns, 0.1-200 microns, 0.1-150 microns, 0.1-100 microns, 0.1-10 microns, 0.1-5 microns, 0.1-2 microns, 0.1-1.5 microns, or 0.1-1 microns.

In another aspect, the present disclosure is directed to articles (useful objects) containing any of the polymeric compositions described above. In one embodiment, the article is constructed entirely of the polymeric composition. In another embodiment, the article contains a coating of the polymeric composition described above, wherein the coating partly or entirely covers one or more (or all) surfaces of the article, particularly surfaces expected to make contact with a bodily part, particularly the hands or fingers. The article may be, for example, a door knob, handle, counter, video screen, tool, filtration membrane, or protective suit.

In another aspect, the present disclosure is directed to a method for inactivating microbes and viruses on a surface of an object by incorporating any of the polymeric compositions described above into a surface of the object. In a first embodiment, the polymeric composition is incorporated into a surface of the object by constructing the object (e.g., fibers, or a mat or membrane constructed of such fibers) entirely of the polymeric composition. A prime example of the latter embodiment is the construction of a filtration membrane from bound or unbound fibers (woven or non-woven and solid or hollow) composed of any of the polymeric compositions described above. The filtration membrane may be useful for filtering or cleansing a liquid or gaseous sample. The filtration membrane is necessarily porous to permit passage of liquid or gas. In another embodiment, the polymeric composition is incorporated into a surface of the object by coating the polymeric composition onto the object by any of the means known in the art for coating of polymers. In the coating process, a partly uncured polymeric composition or mixture of precursor components (e.g., diol or polyol component, isocyanate component, and optionally, a diamino or polyamino component) may be deposited onto the surface of an object and cured by, for example, resting over time at room temperature or an elevated temperature, or by exposure to an energetic electromagnetic source, if appropriate. Alternatively, the cured or partly cured polymeric composition or uncured mixture of components may be dissolved or suspended in a suitable solvent, followed by coating of the object (e.g., by dipping. spraying, spin coating, or the like) with the solution or suspension, followed by removal of the solvent. For example, a filtration membrane constructed of metal or a ceramic may be coated with the polymeric composition by the above method rather than constructing the filtration membrane entirely of the polymeric composition.

### BRIEF DESCRIPTION OF THE FIGURES

FIGS. 1A-1H. Design, fabrication and characterization of the anti-viral membrane (AVM). FIG. 1A is a schematic illustration of the two polyurethanes, HAPU and SBPU. FIG. 1B is a photograph of the membrane made of HAPU and SBPU. FIG. 1C is a scanning electron microscope (SEM) image of the membrane made of HAPU and SBPU before chlorination. FIG. 1D is a schematic illustration of respiratory droplets on smooth. micro fibrous, hydrophobic sub-micro fibrous, and hydrophilic sub-micro fibrous membranes. FIG. 1E is a graph showing immobilized chlorine content (weight percentage) of AVM and cast film as a function of chlorination time. FIG. IF shows X-ray photoelectron spectroscopy (XPS) Cl 2p spectra of the membranes made of HAPU and SBPU, before and after chlorination. FIG. 1G shows stress-strain curves in tensile test for the membranes made of HAPU and SBPU, before and after chlorination. FIG. 1H shows digital photographs of water droplets on AVM and HAPU membranes.

FIG. 2. Schematic illustration for the design and fabrication of the anti-viral membrane (AVM) made of the polymeric composition shown in FIG. 1A.

FIGS. 3A-3E. Data showing that the AVM rapidly and efficiently inactivates a broad spectrum of bacteria. FIG. 3A is graph showing viable E. coli on AVM, PPN, HAPU, as well as PU-N⁺ membranes after different contact time. FIG. 3B is a graph showing stability of immobilized chlorine (weight percentage) on AVM under dry or aqueous condition. FIG. 3C is a graph showing immobilized chlorine content on AVM after 10 chlorination-dechlorination cycles. FIG. 3D is a graph showing viable E. coli on the AVM with different shelf times or recharged for the 5th time. FIG. 3E presents data showing bactericidal efficacies of AVM against S. aureus, VRE, MRSA and the bacterial cocktail after 1 min of contact, with PPN as a control.

FIGS. 4A-4E. Data showing that the AVM rapidly and efficiently inactivates non-enveloped feline calicivirus (FCV) and enveloped transmissible gastroenteritis coronavirus (TGEV). FIG. 4A is a graph showing FCV titer on AVM or PPN control after 2 h, 30 min, 10 min, and 1 min contact. FIG. 4B is a graph showing FCV titer on AVM, HAPU or PU-N⁺ after 2 h, 30 min and 10 min contact. FIG. 4C is a graph showing FCV titer on AVM with different shelf times or recharged for the 5th time after 10 min contact. FIG. 4D is a graph showing (TGEV) titer on AVM or PPN control after 2 h, 30 min, 10 min, and 1 min contact. FIG. 4E is a graph showing TGEV titer on AVM with different shelf times or recharged for the 5th time after 10 min contact. LOD: limit of detection for the TCID₅₀ assay. n=3 per group. ***p-value<0.001, and ****p-value<0.0001.

FIGS. 5A-5C. Data showing that the AVM rapidly and efficiently inactivates SARS-COV-2. FIG. 5A is a graph showing SARS-COV-2 titer on AVM or PPN membranes after 2 h contact. FIG. 5B is a graph showing SARS-COV-2 titer on AVM or PPN membranes after 30 min contact. FIG. 5C is a graph showing SARS-COV-2 titer on AVM or PPN membranes after 10 min contact. n=3 per group. ****p-value<0.0001.

### DETAILED DESCRIPTION OF THE INVENTION

In a first aspect, the present disclosure is directed to a polyurethane composition that includes the following functional groups: (i) a five-membered or six-membered heterocyclic ring containing at least one ring heteroatom selected from nitrogen and oxygen atoms, and (ii) a zwitterionic group. The term “polyurethane” indicates the presence of carbamate (urethane) linkages as a result of the reaction between diols and diisocyanates. However, as the polyurethane composition can include ester and urea linkages, the polyurethane composition is also herein referred to as a “polymeric” composition, wherein it is understood that the polymeric composition includes a polyurethane component. The polyurethane composition may be a product of the reaction between diols and diisocyanates, wherein the diols may contain functional groups (i) and (ii). The polyurethane composition may further include the product of reaction between an ether-containing diol (e.g., PCL-diol) and a diisocyanate, and/or the product of reaction between a diamine (e.g., an alkylenediamine) and a diisocyanate, the latter of which necessarily results in urea linkages. In embodiments, the functional groups (i) and (ii) are pendant groups attached to the polymer backbone. In some embodiments, functional groups (i) and/or (ii) is/are part of the backbone of the polymer. In some embodiments, a functional group (i), such as a hydantoin group or derivative thereof, is a pendant group, while a functional group (ii), such as a sulfobetaine or carboxybetaine, is a part of the backbone of the polymer.

In one embodiment, the polyurethane composition contains first and second polyurethanes (i.e., first and second separate polymer strands) in homogeneous admixture (i.e., as a blend), wherein the first polyurethane contains functional group (i) and not functional group (ii), and the second polyurethane contains functional group (ii) and not functional group (i). In another embodiment, the polyurethane composition contains a single polyurethane (i.e., single polymer strand) containing functional groups (i) and (ii) on the same strand. In some embodiments, the polyurethane contains at least one polyester linkage, at least one polyether linkage, or at least one urea linkage, or multiple numbers of any one of these, or a combination of any one of these linkages. As the composition is a polyurethane composition, the composition typically includes a multiplicity of carbamate linkages. In some embodiments, the polyurethane includes urea linkages (i.e., aside from carbamate linkages).

In some embodiments, functional group (i), also denoted by the group “R”, is a pendant group on the polymer backbone. The foregoing embodiment can be generally depicted by the following formula:

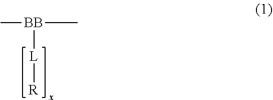

wherein “BB” represents the polymer backbone, L represents a bond or linker, R is the heterocyclic ring of functional group (i), and the subscript x (the number of L-R pendant groups attached to the backbone) is typically at least 1, 2, or 3, and more typically at least or greater than 10, 20, 30, 40, 50, 100, 200, 500, or 1,000. The linker may be, for example, an alkylene linker, carbonyl linker, amide linker, urea linker, ether-containing linker, carbamate linker, carbonate linker, or a linker containing a combination of any two or more of these linkers connected to each other.

In other embodiments, functional group (i) is within the polymer backbone. The foregoing embodiment can be generally depicted by the following formula:

-BB-R-BB- (2)

wherein R is the heterocyclic ring of functional group (i), shown embedded within the polymer backbone BB. When the heterocyclic ring is within the polymer backbone, the heterocyclic ring necessarily functions as a linker, i.e., to link different portions of the polymer backbone, wherein each instance of “BB” represents a different portion of the polymer backbone attached to the heterocyclic ring (R). The heterocyclic ring can be attached to two BB moieties by two of its ring carbon atoms, two of its ring nitrogen atoms, or a ring carbon atom and ring nitrogen atom. Formula (2) is intended as a repeating unit in the polymer; thus, R can be present in the polymer in any of the same numbers provided above for x.

Functional group (i) is a five-membered or six-membered heterocyclic ring containing at least one ring heteroatom selected from nitrogen and oxygen atoms. The heterocyclic ring may contain only nitrogen ring heteroatoms, only oxygen ring heteroatoms, or both types of ring heteroatoms only. In some embodiments, the heterocyclic ring contains precisely or at least two ring heteroatoms selected from only nitrogen atoms, only oxygen atoms, or both nitrogen and oxygen atoms. The heterocyclic ring may also be saturated or unsaturated. The heterocyclic ring may or may not include one or more oxo (carbonyl) atoms on the ring. The presence of one or more oxo groups on the ring qualifies the ring as unsaturated. The heterocyclic ring is attached via one of its ring carbon or nitrogen atoms as a pendant group on the polymer backbone. In one embodiment, the heterocyclic ring is directly attached to the polymer backbone by one of its ring carbon or nitrogen atoms. In another embodiment, the heterocyclic ring is indirectly attached to the polymer backbone by a linker, such as an alkylene linker of the formula —(CH₂)ₙ— wherein n is 1, 2, or 3, wherein the linker connects the polymer backbone and a ring carbon or nitrogen atom of the heterocyclic ring. Notably, for purposes of the invention, the heterocyclic ring is uncharged (i.e., neutral) when attached to the polymer.

Aside from the ring heteroatoms, the five-membered or six-membered heterocyclic ring may or may not contain one or more substituents, such as one or more of lower alkyl (e.g., C₁₋₃, such as methyl, ethyl, n-propyl, or isopropyl), carbonyl group, hydroxy group, ether group, amino group, carboxylic acid group, or amide group. In specific embodiments, functional group (i) is or includes hydantoin (imidazolidine-2,4-dione) or a hydantoin derivative (e.g., 5,5-dimethyl-2,4-dione or N-methyl-2,4-dione). In some embodiments, the heterocyclic ring contains one or more alkyl substituents, typically containing 1, 2, 3, or 4 carbon atoms (e.g., methyl, ethyl, n-propyl, isopropyl, n-butyl, sec-butyl, isobutyl, or t-butyl), on one or more ring carbon atoms and/or ring nitrogen atoms.

In one set of embodiments, functional group (i) is a saturated five-membered or six-membered heterocyclic ring containing precisely or at least one, two, or three ring nitrogen atoms and no ring oxygen atoms. Some examples of saturated five-membered or six-membered heterocyclic rings containing precisely or at least one, two, or three ring nitrogen atoms and no ring oxygen atoms include pyrrolidine, piperidine, piperazine, and imidazolidine rings.

In another set of embodiments, functional group (i) is an unsaturated five-membered or six-membered heterocyclic ring containing precisely or at least one, two, or three ring nitrogen atoms and no ring oxygen atoms. Some examples of unsaturated five-membered or six-membered heterocyclic rings containing precisely or at least one, two, or three ring nitrogen atoms and no ring oxygen atoms include pyrrole, imidazole, 2-oxo-imidazolidine, 4-oxo-imidazolidine, imidazolidine-2,4-dione (hydantoin), 5,5-dimethyl-imidazolidine-2,4-dione (5,5-dimethylhydantoin), pyrazole, pyridine, pyrazine, pyrimidine, and 1,3,5-triazine rings. In particular embodiments, functional group (i) is a bydantoin group or derivative thereof (examples of which are provided above), typically connected to the polymer backbone by its ring nitrogen atom at the 3-position of the hydantoin ring, while the ring carbon atom at the 5-position of the hydantoin ring may or may not be substituted with one or two alkyl groups (typically containing 1, 2, or 3 carbon atoms), e.g., one or two methyl and/or ethyl groups.

In another set of embodiments, functional group (i) is a saturated five-membered or six-membered heterocyclic ring containing precisely or at least one, two, or three ring oxygen atoms and no ring nitrogen atoms. Some examples of saturated five-membered or six-membered heterocyclic rings containing precisely or at least one, two, or three ring oxygen atoms and no ring nitrogen atoms include tetrahydrofuran, tetrahydropyran, 1,4-dioxane, 1,3-dioxane, gamma-butyrolactone, 2-furanone, succinic anhydride, and maleic anhydride rings.

In another set of embodiments, functional group (i) is an unsaturated five-membered or six-membered heterocyclic ring containing precisely or at least one, two, or three ring oxygen atoms and no ring nitrogen atoms. Some examples of unsaturated five-membered or six-membered heterocyclic rings containing precisely or at least one, two, or three ring oxygen atoms and no ring nitrogen atoms include furan and pyran rings.

In another set of embodiments, functional group (i) is a saturated five-membered or six-membered heterocyclic ring containing precisely or at least one, two, or three ring nitrogen atoms and precisely or at least one oxygen atom. Some examples of saturated five-membered or six-membered heterocyclic rings containing precisely or at least one, two, or three ring nitrogen atoms and precisely or at least one oxygen atom include morpholine and oxazolidine rings.

In another set of embodiments, functional group (i) is an unsaturated five-membered or six-membered heterocyclic ring containing precisely or at least one, two, or three ring nitrogen atoms and precisely or at least one oxygen atom. Some examples of unsaturated five-membered or six-membered heterocyclic rings containing precisely or at least one, two, or three ring nitrogen atoms and precisely or at least one oxygen atom include oxazole, isoxazole, oxazoline, 1,2,5-oxadiazole (furazan), 1,3,4-oxadiazole, 2-oxazolidinone, 4-oxazolidinone, 5-oxazolidinone, 2,4-oxazolidinedione, and 2,5-oxazolidinedione rings.

Functional group (ii) is a zwitterionic group. As well known, a zwitterionic group is a group simultaneously possessing a positive and negative charge, wherein the positive and negative charges are bound within the group. The zwitterionic group can be any zwitterionic group known in the art, such as a betaine group, or more specifically, a sulfobetaine, carboxybetaine, or phosphobetaine group. In particular embodiments, the zwitterionic group is a sulfobetaine group, such as n-tetradecyl-N,N-dimethyl-3-ammonio-1-propanesulfonate, n-octyl-N,N-dimethyl-3-ammonio-1-propanesulfonate, n-decyl-N,N-dimethyl-3-ammonio-1-propanesulfonate, and n-dodecyl-N,N-dimethyl-3-ammonio-1-propanesulfonate. The foregoing zwitterionic groups may be pendant groups attached to the backbone of the polymer. Alternatively, the zwitterionic group is incorporated into (i.e., embedded in) the backbone of the polymer.

In some embodiments, functional group (ii), also denoted by the group “Z”, is a pendant group on the polymer backbone. The foregoing embodiment can be generally depicted by the following formula:

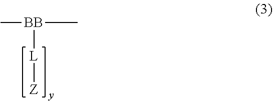

wherein “BB” represents the polymer backbone, L represents a bond or linker, Z is the zwitterionic group of functional group (ii), and the subscript y (the number of L-Z pendant groups attached to the backbone) is typically at least 1, 2, or 3, and more typically at least or greater than 10, 20, 30, 40, 50, 100, 200, 500, or 1,000. The linker may be, for example, an alkylene linker, carbonyl linker, amide linker, urea linker, ether-containing linker, carbamate linker, carbonate linker, or a linker containing a combination of any two or more of these linkers connected to each other.

In particular embodiments of Formula (3), the pendant Z portion is a pendant betaine zwitterionic group. The pendant betaine may be, for example, a pendant ammonium betaine, pendant phosphonium betaine, or phosphate betaine, as discussed in greater detail below.

In some embodiments, the pendant betaine is a pendant ammonium betaine. The pendant ammonium betaines can be generally depicted by the following formula:

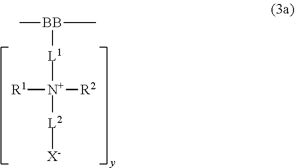

wherein “BB” represents the polymer backbone, L¹ and L² are independently selected from a bond or linker, y is as defined above, and R¹ and R² are hydrocarbon (typically linear or branched alkyl) groups, independently containing 1-20 (or e.g., 1-16, 1-12, 1-8, 1-6, 1-4, or 1-3) carbon atoms. Each of L¹ and L² may independently be selected from, for example, a bond, an alkylene linker, carbonyl linker, amide linker, urea linker, ether-containing linker, carbamate linker, carbonate linker, or a linker containing a combination of any two or more of these linkers connected to each other. The group X⁻ represents a negatively-charged group, such as a sulfonate group, carboxylate group, or phosphonate group. When X⁻ is sulfonate (—SO₃⁻), the ammonium betaine is sulfobetaine. When X⁻ is carboxylate (—CO₂⁻), the ammonium betaine is a carboxybetaine. When X⁻ is a phosphonate (—P(O)₃H⁻), the ammonium betaine is phosphobetaine.

Notably, the positively charged nitrogen atom in Formula (3a) may be replaced with a positively charged phosphorus atom, with all else the same, to result in corresponding phosphonium betaines, such as depicted by the following formula:

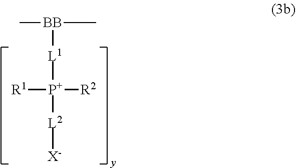

The pendant zwitterion may alternatively be a phosphate betaine. The phosphate betaines can be generally depicted by the following formula:

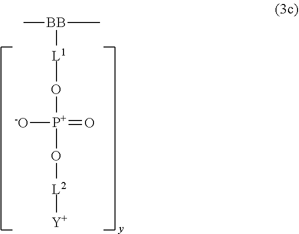

wherein “BB” represents the polymer backbone, y is as defined above, and L¹ and L² are independently selected from a bond or linker. Each of L¹ and L² may independently be selected from, for example, a bond, an alkylene linker, carbonyl linker, amide linker, urea linker, ether-containing linker, carbamate linker, carbonate linker, or a linker containing a combination of any two or more of these linkers connected to each other. The group Y⁺ represents a positively-charged group, such as an ammonium or phosphonium group.

In other embodiments, functional group (ii) is within the polymer backbone. The foregoing embodiment can be generally depicted by the following formula:

-BB-Z-BB- (4)

wherein Z is the zwitterionic group of functional group (ii), shown embedded within the polymer backbone BB. When the zwitterionic group is within the polymer backbone, the zwitterionic group necessarily functions as a linker, i.e., to link different portions of the polymer backbone, i.e., the wherein each instance of “BB” represents a different portion of the polymer backbone attached to the zwitterionic group (Z). Formula (4) is intended as a repeating unit in the polymer; thus. Z can be present in the polymer in any of the same numbers provided above for y.

The backbone zwitterionic group may be a backbone betaine zwitterionic group. The backbone betaine may be, for example, a backbone ammonium betaine, backbone phosphonium betaine, or backbone phosphate betaine, as discussed in greater detail below.

The backbone betaine may be a backbone ammonium betaine. The backbone ammonium betaine can be generally depicted by the following formula:

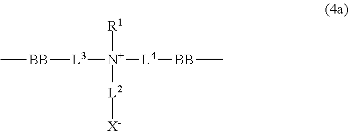

wherein “BB” represents the polymer backbone, L², L³, and L⁴ are independently selected from a bond or linker, and R¹ is a hydrocarbon (typically linear or branched alkyl) group, typically containing 1-20 (or e.g., 1-16, 1-12, 1-8, 1-6, 1-4, or 1-3) carbon atoms. Each of L², L³, and L⁴ may independently be selected from, for example, a bond, an alkylene linker, carbonyl linker, amide linker, urea linker, ether-containing linker, carbamate linker, carbonate linker, or a linker containing a combination of any two or more of these linkers connected to each other. The group X⁻ represents a negatively-charged group, such as a sulfonate group, carboxylate group, or phosphonate group. The above formula represents only one portion of the polyurethane polymer in which the ammonium betaine is located, and this portion likely repeats through the polymer; thus, the ammonium betaine is typically present in large numbers (such as given by x or y) in the polyurethane polymer.

The backbone betaine may alternatively be a backbone phosphonium betaine. The backbone phosphonium betaine can be generally depicted by the following formula:

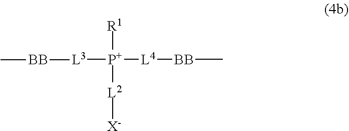

wherein “BB” represents the polymer backbone, L², L³, and L⁴ are independently selected from a bond or linker, and R¹ is a hydrocarbon (typically linear or branched alkyl) group, typically containing 1-20 (or e.g., 1-16, 1-12. 1-8, 1-6, 1-4, or 1-3) carbon atoms. Each of L², L³, and L⁴ may independently be selected from, for example, a bond, an alkylene linker, carbonyl linker, amide linker, urea linker, ether-containing linker, carbamate linker, carbonate linker, or a linker containing a combination of any two or more of these linkers connected to each other. The group X⁻ represents a negatively-charged group, such as a sulfonate group, carboxylate group, or phosphonate group. The above formula represents only one portion of the polyurethane polymer in which the phosphonium betaine is located, and this portion likely repeats through the polymer; thus, the phosphonium betaine is typically present in large numbers (such as given by x or y) in the polyurethane polymer.

The backbone betaine may alternatively be a backbone phosphate betaine. The backbone phosphate betaine can be generally depicted by the following formula:

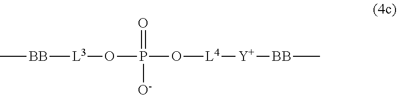

wherein “BB” represents the polymer backbone and L³ and L⁴ are independently selected from a bond or linker. Each of L³ and L⁴ may independently be selected from, for example, a bond, an alkylene linker, carbonyl linker, amide linker, urea linker, ether-containing linker, carbamate linker, carbonate linker, or a linker containing a combination of any two or more of these linkers connected to each other. The group Y⁺ represents a positively-charged group, such as an ammonium or phosphonium group. The above formula represents only one portion of the polyurethane polymer in which the phosphate group and Y⁺ are located, and this portion likely repeats through the polymer; thus, the phosphate group and Y⁺ are each typically present in large numbers (such as given by x or y) in the polyurethane polymer.

In some embodiments, functional groups (i) and (ii), i.e., R and Z, are both pendant groups attached to the same polymer backbone. The foregoing embodiment can be generally depicted by the following formula:

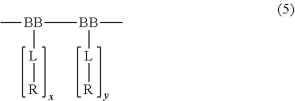

wherein “BB” represents the polymer backbone. L in each instance independently represents a bond or linker, R is the heterocyclic ring of functional group (i), Z is the zwitterionic group of functional group (ii), subscript x (the number of L-R pendant groups attached to the backbone) is typically at least 1, 2, or 3, and more typically a high multiplicity, e.g., at least 10, 20, 30, 40, 50, or 100, and the subscript y (the number of L-Z pendant groups attached to the backbone) is typically at least 1, 2, or 3, and more typically a high multiplicity, e.g., at least 10, 20, 30, 40, 50, or 100. The linker may independently be, for example, an alkylene linker, carbonyl linker, amide linker, urea linker, ether-containing linker, carbamate linker, carbonate linker, or a linker containing a combination of any two or more of these linkers connected to each other.

In other embodiments, functional group (i) (i.e., R) is pendant on the polymer backbone while functional group (ii) (i.e., Z) is embedded within the same polymer backbone. The foregoing embodiment can be generally depicted by the following formula:

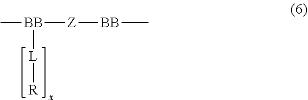

wherein “BB” represents the polymer backbone, L represents a bond or linker, R is the heterocyclic ring of functional group (i), Z is the zwitterionic group of functional group (ii), and subscript x (the number of L-R pendant groups attached to the backbone) is typically at least 1, 2, or 3, and more typically a high multiplicity, e.g., at least 10, 20, 30, 40, 50, or 100. The linker may be, for example, an alkylene linker, carbonyl linker, amide linker, urea linker, ether-containing linker, carbamate linker, carbonate linker, or a linker containing a combination of any two or more of these linkers connected to each other. The above formula represents only one portion of the polyurethane polymer in which R and Z are located, and this portion likely repeats through the polymer, thus, R and Z are each typically present in large numbers (such as given by x and y, respectively) in the polyurethane polymer.

In other embodiments, functional group (i) (i.e., R) is embedded within the polymer backbone while functional group (ii) (i.e., Z) is pendant on the same polymer backbone. The foregoing embodiment can be generally depicted by the following formula:

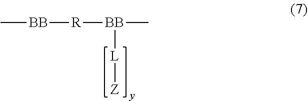

wherein “BB” represents the polymer backbone, L represents a bond or linker, R is the heterocyclic ring of functional group (i), Z is the zwitterionic group of functional group (ii), and subscript y (the number of L-Z pendant groups attached to the backbone) is typically at least 1, 2, or 3, and more typically a high multiplicity, e.g., at least 10, 20, 30, 40, 50, or 100. The linker may be, for example, an alkylene linker, carbonyl linker, amide linker, urea linker, ether-containing linker, carbamate linker, carbonate linker, or a linker containing a combination of any two or more of these linkers connected to each other. The above formula represents only one portion of the polyurethane polymer in which R and Z are located, and this portion likely repeats through the polymer; thus, R and Z are each typically present in large numbers (such as given by x and y, respectively) in the polyurethane polymer.

In other embodiments, functional groups (i) and (ii), i.e., R and Z, are both embedded within the same polymer backbone. The foregoing embodiment can be generally depicted by the following formula:

-BB-R-BB-Z-BB- (8)

wherein “BB” represents the polymer backbone, R is the heterocyclic ring of functional group (i), and Z is the zwitterionic group of functional group (ii). The above formula represents only one portion of the polyurethane polymer in which R and Z are located, and this portion likely repeats through the polymer; thus, R and Z are each typically present in large numbers (such as given by x and y, respectively) in the polyurethane polymer.

In some embodiments, the polymeric composition contains a halamine group, which contains a nitrogen atom attached to a halogen atom. The halogen atom may be a chlorine, bromine, or iodine atom, more typically a chlorine atom. The nitrogen atom typically belongs to a primary amine (—NH₂) or secondary amine (e.g., —NH—) before being converted to the halamine (by replacement of the hydrogen atom in the amine with the halogen atom, such as chlorine). In some embodiments, the nitrogen atom coordinated to the halogen (e.g., chlorine) atom may be a nitrogen atom in functional group (i), e.g., a ring nitrogen atom or amino-containing group in functional group (i). In other embodiments, the nitrogen atom coordinated to the halogen (e.g., chlorine) atom may be located in a urethane linkage, urea linkage, or a pendant amino-containing group in the polyurethane. In some embodiments, the halamine is present in both functional group (i) and in a linkage (e.g., urethane or urea linkage) of the polymer. In an exemplary embodiment, the halamine is a chlorine atom or other halogen atom coordinated to a ring nitrogen atom of functional group (i). In further specific embodiments, the halamine is present in functional group (i), wherein functional group (i) is hydantoin or a derivative thereof, as described in detail earlier above. The chlorine or other halogen atom provides the polymeric composition with additional ability to inactivate a microbe or virus.

In another aspect, the present disclosure is directed to a method of making the anti-microbial polymeric composition described above. As the polymeric composition contains a polyurethane component, the polymeric composition includes at least a product of reaction between diols and diisocyanates. The reaction conditions by which diols and diisocyanates react to form a polyurethane is well known in the art. For example, the diol and diisocyanate may be combined in an anhydrous polar inert solvent, such as DMSO, and heated to a temperature of 60-90° C. in the presence of a suitable catalyst, such as stannous octoate, i.e., Sn(Oct)₂, wherein octoate is 2-ethylhexanoate Typically, to incorporate functional groups (i) and (ii) into the polymer, diol-derivatized versions of functional groups (i) and (ii) are reacted with the diisocyanate. For example, where R represents functional group (i), a diol molecule of the formula HO—R—OH can be reacted with the diisocyanate to form an R-containing portion of the polymer. Similarly, where Z represents functional group (ii), a diol molecule of the formula HO—Z—OH can be reacted with the diisocyanate to form a Z-containing portion of the polymer. In some embodiments, the polyurethane composition may further include the product of reaction between an ether-containing diol and a diisocyanate, wherein the ether-containing diol may be represented by the formula HO-[PAG]-OH, wherein the PAG (polyalkylene glycol) may be or include a polyethylene glycol (PEG) segment, and wherein the ether-containing diol may additionally or alternatively include one or more alkylene segments (i.e., —(CH₂)_{z}-segments, where z=1, 2, 3, 4, 5, 6, 7, 8, 9, or 10, or a range therein, e.g., 1-10 or 1-6), and the ether-containing diol may or may not include one or more ester or amide linkages. In specific embodiments, the ether-containing diol is the product of a ring opening polymerization of a lactone, such as caprolactone (CL), which may, in some embodiments, include a diol initiator, such as ethylene glycol, diethylene glycol, or triethylene glycol. The result of the ring opening polymerization of CL is polycaprolactone (PCL), which is a diol, and thus, can be included in the reaction to react with the diisocyanate and become incorporated in the polymer. In some embodiments, the polyurethane composition may further or alternatively include the product of reaction between a diamine and a diisocyanate to result in urea linkages, wherein the diamine may contain primary or secondary amine functional groups. In particular embodiments, the diamine is an alkylenediamine, such as one having the formula H₂N—(CH₂)_{z}—NH₂, wherein z is independently selected from any of the values provided above. In specific embodiments, the diamine is a diaminobutane, such as 1,4-diaminobutane. Typically, a viscous polymer solution is formed once the reaction is complete, and the viscous polymer can be precipitated by addition of a non-polar solvent (e.g., toluene or hexanes), removed from the solution, washed, and dried.

The diisocyanate can be any of the diisocyanates known in the art. Some examples of diisocyanates include 1,6-hexamethylene diisocyanate (HDI), 4,4′-methylene diphenyl diisocyanate (MDI), 4,4′-diisocyanato dicyclohexylmethane (hydrogenated MDI), and isophorone diisocyanate (IPDI). Any one or combination of the foregoing diisocyanates may be included in the reaction and become incorporated into the polymer. Thus, any one or combination of the foregoing diisocyanates may be reacted with any one or more diols (and optionally diamine) described above and thereby become incorporated into any one of the polymeric compositions described above.

In some embodiments, the polymeric composition or an article made thereof (e.g., membrane) is porous. The term “porous” indicates the presence of pores. For example, the polymeric composition can be made into fibers, and the fibers assembled into a fibrous structure, which is porous. The pores are typically, on average, at least 0.2 microns and up to 10 microns. In different embodiments, the pores in the polymeric composition or article made thereof are precisely or at least 0.1, 0.2, 0.5, 1, 1.5, 2. 2.5, 3, 3.5, 4, 5, 6, 7, 8, 9, or 10 microns, or a pore size within a range bounded by any two of the foregoing values, e.g., 0.1-10 microns, 0.1-5 microns, 0.1-3 microns, 0.1-2 microns, 0.2-10 microns, 0.2-5 microns, 0.2-3 microns, 0.2-2 microns, 0.5-10 microns, 0.5-5 microns, 0.5-3 microns, 0.5-2 microns, 1-3 microns, 1-2 microns, 1.5-3 microns, and 1.5-2 microns.

The polymeric composition may also be made to assume any of a variety of shapes, including a film or membrane shape or structure. The shape may be made by, for example, pouring the partially reacted polymer into a mold followed by curing and casting an object; injection molding; or an additive manufacturing (AM) process, e.g., extrusion or binder jetting AM process. In the case of a film or membrane, the film or membrane may function as a coating on an underlying object for which microbial or viral resistance is desired. In some embodiments, the polymeric composition is porous. In other embodiments, the polymeric composition is non-porous. In some embodiments, the polymeric composition has a fibrous structure, i.e., it is constructed of fibers that are bonded to each other, wherein the fibers may be woven or non-woven and may be solid or hollow. The fibers can be produced by any suitable method, such as melt spinning or electrospinning. In various embodiments, the fibers may have a diameter within a range of 0.1-1000 microns, 0.1-500 microns, 0.1-200 microns, 0. 1-150 microns, 0.1-100 microns, 0.1-10 microns, 0.1-5 microns, 0.1-2 microns, 0.1-1.5 microns, 0.1-1 microns, 0.2-2 microns, 0.2-1.5 microns, or 0.2-1 microns.

In another aspect, the present disclosure is directed to articles (useful objects) constructed of or containing any of the polymeric compositions described above. In one embodiment, the article is constructed entirely of the polymeric composition. In another embodiment, the article contains a coating of the polymeric composition described above. wherein the coating partly or entirely covers one or more (or all) surfaces of the article, particularly surfaces expected to make contact with a bodily part, such as the hands or fingers. The article may be, for example, a door knob, handle, counter, video screen, tool (e.g., medical device), filtration membrane, or protective suit. The object may also be one designed to enter the body, e.g., an endoscope (e.g., for upper, gastrointestinal, or nasal endoscopy), surgery protective (puncture resistant) film, or glaucoma drainage device. The object being coated with the polymeric composition may be any of the foregoing objects.

In another aspect, the present disclosure is directed to a method for inactivating microbes and/or viruses on a surface of an object by incorporating any of the polymeric compositions described above onto or into a surface of the object. In a first embodiment, the polymeric composition is incorporated into a surface of the object by constructing the object (e.g., fibers, or a mat or membrane constructed of such fibers) entirely of the polymeric composition. A prime example of the latter embodiment is the construction of a filtration membrane from bound or unbound fibers (woven or non-woven and solid or hollow) composed of any of the polymeric compositions described above. The filtration membrane may be useful for filtering or cleansing a liquid or gaseous sample. The filtration membrane is necessarily porous to permit passage of liquid or gas. In another embodiment, the polymeric composition is incorporated into a surface of the object by coating the polymeric composition onto the object by any of the means known in the art for coating of polymers. In the coating process, a partly uncured polymeric composition or mixture of precursor components (e.g., diol or polyol component, isocyanate component, and optionally, a diamino or polyamino component) may be deposited onto the surface of an object and cured by, for example, resting over time at room temperature or an elevated temperature, or by exposure to an energetic electromagnetic source, if appropriate. Alternatively, the cured or partly cured polymeric composition or uncured mixture of components may be dissolved or suspended in a suitable solvent, followed by coating of the object (e.g., by dipping, spraying, spin coating, or the like) with the solution or suspension, followed by removal of the solvent. For example, a filtration membrane constructed of metal or a ceramic may be coated with the polymeric composition by the above method rather than constructing the filtration membrane entirely of the polymeric composition.

Examples have been set forth below for the purpose of illustration and to describe the best mode of the invention at the present time. However, the scope of this invention is not to be in any way limited by the examples set forth herein.

### EXAMPLES

### Overview

Herein is reported a safe, inexpensive and scalable membrane with covalently immobilized chlorine, a large surface area, and fast wetting. The membrane exhibits long-lasting, exceptional killing efficacy against a broad spectrum of bacteria and viruses. The membrane can achieve more than 6 log reduction within several minutes against all five bacterial strains tested, including gram-positive, gram-negative, and drug-resistant types, as well as a clinical bacterial cocktail. The membrane also efficiently deactivated non-enveloped and enveloped viruses in minutes. In particular, a 5.17 log reduction was achieved against SARS-COV-2 after only 10 minutes of contact with the membrane. This membrane may be used on high-touch surfaces in healthcare and other public facilities or in air filters and personal protective equipment to provide continuous protection and minimize transmission risks.

The anti-viral membrane (AVM) developed herein preferably possesses three desirable attributes: (1) a potent and safe antiviral agent that can be stably incorporated into the membrane; (2) a large surface to volume ratio to maximize the immobilization of the antiviral agent on the surface of the membrane; and (3) high wettability to promote rapid and intimate contact with viral contaminants for effective contact killing. To develop such an AVM, two miscible polyurethanes were designed and synthesized, one with a hydantoin side group which can covalently bond and stably immobilize oxidative chlorine and one with a zwitterionic group that imparts hydrophilicity and promotes fast wetting. Electrospinning was then used to make the sub-micron fibrous membrane from the polyurethane blends. The sub-micron fibrous structures provide a large surface area (˜10 m²/g) for chlorine immobilization and also enhance wetting even for small size, respiratory droplets. To prepare the membrane for use, it was treated with a household chlorine-based disinfectant, rinsed to remove any free chlorine, and dried.

The AVM exhibits near complete killing with 99.9999% reduction within 1 min of all five bacterial strains tested, including gram-positive, gram-negative and drug-resistant ones as well as a clinical bacterial cocktail. The AVM was also tested against enveloped viruses, transmissible gastroenteritis coronavirus (TGEV) and SARS-COV-2, and non-enveloped feline calicivirus (FCV). No viable TGEV was detectable after just 1 min exposure. Although the non-enveloped FCV was somewhat more resistant, no infectious virus was detectable after 30 minutes; and 1 and 10 minutes sufficed to reduce infectivity by 2.17 and 4.72 log, respectively. In particular, a 5.17 log reduction of SARS-COV-2 was achieved after a 10 minutes of contact, and no infectious SARS-COV-2 was detectable after 30 minutes. The AVM may be attached to high-touch surfaces or used in air filters and personal protective equipment (PPE) to provide continuous protection and minimize transmission risks.

Polycaprolactone diol (Mₙ 2000, PCL-diol) was dried in a vacuum oven prior to synthesis. 5,5-dimethylhydantoin, diethanolamine, formaldehyde solution (36.0% in H₂O), stannous octoate (Sn(Oct)₂), 1.4-diaminobutane, sodium dichloroisocyanurate (NaDCC), potassium iodine, anhydrous dimethyl sulfoxide (DMSO), dichloromethane (DCM), diethyl ether, methanol, N-butyldiethanolamine, 1,3-propanesultone, 1,6-diisocyanatohexane (HDI), and 1,1,1,3,3,3-hexafluoro-2-propanol (HFIP) were commercially obtained. PCL-diol, Sn(Oct)₂, and 1,4-diaminobutane were dried in a vacuum oven prior to synthesis for removal of residual water.

### Cells, Bacteria, and Viruses

Swine testicular (ST; ATCC CRL-1746) cells, Crandell-Rees Feline Kidney (CRFK; ATCC CCL-94) cells, and African green monkey kidney (Vero E6; ATCC NR-596) cells were purchased from the American Type Culture Collection (ATCC). Bacterial strains Staphylococcus aureus (S. aureus; ATCC 6538), Escherichia coli (E. coli; CFT-73), methicillin-resistant Staphylococcus aureus (MRSA; US300), vancomycin-resistant Enterococci (VRE; clinical isolate), and a 14-strain bacterial cocktail were obtained from the Halomine. Inc. Transmissible gastroenteritis virus (TGEV; ATCC VR-1740) and feline calicivirus (FCV; ATCC VR-782) were purchased from the ATCC. SARS-COV-2 strain USA-WA1/2020 was obtained from BEI Resources (Manassas, VA, USA).

### Material Synthesis

### Synthesis of 3-((bis(2-hydroxyethyl)amino)methyl)-5,5-dimethylimidazolidine-2,4-dione (HA-diol)

5,5-dimethylhydantoin (26.4 g, 0.2 mol), diethanolamine (21.2 g, 0.2 mol), formaldehyde (16.2 g, 0.2 mol), and methanol (250 mL) were added to a 500 mL round-bottom flask. The mixture was stirred under a nitrogen atmosphere for 6 h at room temperature. After stirring, methanol and water byproducts were removed by rotary evaporation. The remaining viscous crude product was then dissolved in ethyl acetate, with anhydrous sodium sulphate added for further drying. After removal of sodium sulfate by filtration, the solution was refrigerated overnight. A white solid precipitate formed, which was collected and washed twice with cold ethyl acetate before further purification via a silica gel column (eluent: DCM/methanol, 9:1 v/v). The chemical structure of the product (HA-diol) was confirmed by proton nuclear magnetic resonance. ¹H NMR (DMSO-d₆, 400 MHz, ppm): δ 8.28 (s, 1H), 4.37 (t, 2H), 4.30 (s, 2H), 3.42 (m, 2H), 2.63 (t, 2H), 1.28 (s, 6H).

### Synthesis of 3-(butylbis(2-hydroxyethyl)ammonio)propane-1-sulfonate (SB-diol)

N-Butyldiethanolamine (8.05 g, 0.05 mol), 1,3-propanesultone (6.7 g, 0.055 mol) and dichloromethane (200 mL) were added to a 500 mL round-bottom flask. The mixture was stirred under a nitrogen atmosphere for 24 h at 40° C. After stirring, the solvent was removed using a rotary evaporator. The product was precipitated by cold diethyl ether and then washed three times with cold diethyl ether to produce a white powder. The chemical structure of the product (SB-diol) was confirmed by proton nuclear magnetic resonance. ¹H NMR (D₂O, 400 MHz, ppm): δ 3.97 (t, 4H), 3.54 (m, 6H), 3.4 (t, 2H), 2.91 (t, 2H), 2.15 (m, 2H), 1.68 (m, 2H), 1.34 (m, 2H), 0.90 (t, 3H).

### Synthesis of N,N-bis(2-hydroxyethyl)-N-methylbutan-1-aminium (N⁺-diol)

N-Butyldiethanolamine (8.05 g, 0.05 mol), iodomethane (7.8 g, 0.055 mol) and anhydrous acetonitrile (250 mL) were added to a 500 mL round-bottom flask. The mixture was stirred under a nitrogen atmosphere for 24 h at 60° C. After mixing, the solvent was removed by a rotary evaporator. The product was precipitated by cold diethyl ether and subsequently washed three times with cold diethyl ether to get a white powder. The chemical structure of the N,N-bis(2-hydroxyethyl)-N-methylbutan-1-aminium (N⁺-diol) was confirmed by proton nuclear magnetic resonance. 1H NMR (D20, 400 MHz, ppm): δ 4.07 (t, 4H), 3.60 (m, 4H), 3.46 (m, 2H), 3.19 (s, 3H), 1.78 (m, 2H), 1.41 (m, 2H), 0.97 (t, 3H).

### Synthesis of Hydantoin (HA) Based Polyurethane (HAPU)

The HAPU was synthesized from PCL-diol, HA-diol, HDI, and 1,4-diaminobutane. Briefly, PCL-diol (10 g, 5 mmol) and HA-diol (3.9 g, 16 mmol) were dissolved in 250 mL anhydrous DMSO at 65° C. under nitrogen protection. HDI (4.04 g, 24 mmol) was then added to the flask dropwise, followed by the addition of two droplets of Sn(Oct)₂ catalyst. The mixture was stirred vigorously at 65° C. for 30 min. Then, 1,4-diaminobutane (0.26 g, 3 mmol) was added dropwise to the solution as a chain extender and stirred at 80° C. for 6 hours. After the reaction, the viscous polymer solution was precipitated in DI water, and then washed three times with DI water. The resulting white powder (HAPU) was dried in vacuo at 60° C.

### Synthesis of Sulfobetaine (SB) Based Polyurethane (SBPU)

The SBPU was synthesized from PCL-diol, SB-diol, HDI, and 1,4-diaminobutane. Briefly, PCL-diol (10 g, 5 mmol) and SB-diol (4.5 g, 16 mmol) were dissolved in 250 mL anhydrous DMSO at 65° C. under nitrogen protection. HDI (4.04 g, 24 mmol) was then added to the flask dropwise, followed by the addition of two droplets of Sn(Oct)₂ catalyst. The mixture was stirred vigorously at 65° C. for 30 min. After mixing, the chain extender 1,4-diaminobutane (0.26 g, 3mmol) was added dropwise to the solution, which was further stirred at 80° C. for 6 h. After the reaction, the viscous polymer solution was precipitated in diethyl ether, and then washed three times with diethyl ether. The resulting white powder (SBPU) was then washed three times with DI water and dried in vacuo at 60° C. overnight.

### Synthesis of Quaternary Amine Based Polyurethane (PU-N⁺)

The PU-N⁺ was synthesized from PCL-diol, N⁺-diol, HDI, and 1,4-diaminobutane. Briefly, PCL-Diol (10 g, 5 mmol) and N⁺-diol (4.85 g, 16 mmol) were dissolved in 250 ml anhydrous DMSO solvent at 65° C. under nitrogen protection. HDI (4.04 g, 24 mmol) was then added to the flask dropwise, followed by two droplets of Sn(Oct)₂ catalyst. The mixture was stirred vigorously at 80° C. for 2 h. The chain extender 1,4-diaminobutane (0.26 g, 3mmol) was then added dropwise to the solution and stirred at 80° C. for 6 h. After the reaction, the viscous polymer solution was precipitated in DI water and then washed three times with DI water. The resulting white powder (PU-N⁺) was placed in vacuum oven at 60° C. overnight to remove the residual solvent.

### Fabrication of the Anti-Viral Membrane (AVM)

The anti-viral membranes were fabricated through an electrospinning process. Briefly, HAPU and SBPU polymers (mass ratio: 3:1) were dissolved in HFIP at 20% (w/v) at room temperature. The polymer solution was then loaded in a 20-mL plastic syringe and injected at 2.4 mL/h by syringe pump. The sub-micron fibers were spun at 15 kV with a 22G blunt needle as the spinneret that was mounted on a robotic arm. The distance between the needle tip and the collector was set to 12 cm. The rotating aluminum rods (rotating speed: 400-450 rpm) or copper plate were covered with aluminum foil and placed in the path of the polymer solution jet to collect the electrospun fibers. After electrospinning, the membranes were peeled off from rod or aluminum foil, washed with DI water three times and dried in vacuo at room temperature to remove any residual solvent. The dried membranes were then cut into small pieces (1×1 inch²) with a weight of 30-34 mg and treated with 10% (v/v) household bleach solution (8.25% hypochlorite, pH adjusted to 7.0 with HCl) for 15 min or with 10% aqueous solution of sodium dichloroisocyanurate (NaDCC) for 20 min to chlorinate the membranes. Subsequently, the chlorinated membranes were thoroughly washed with DI water and then dried to obtain the anti-viral membrane (AVM).

### Determination of Chlorine Content on the AVM

The immobilized chlorine content of AVM was determined using the iodometric/thiosulfate titration method. In brief, the AVM was immersed into 50 mL aqueous solution containing 1 mL of 0.1 mol/L acetic acid and 0.25 g potassium iodide. The mixture was then shaken at room temperature for 30 min to form I₂. Drops of the 0.5% starch indicator solution was added until the sample turned blue. The mixture was titrated with a solution of 0.02 mol/L sodium thiosulfate until the sample turned from blue to colorless. The amount of immobilized chlorine content was calculated using the following formula:

[Cl %]=35.45×(C/V)/2 W>100%

where Cl % is the weight percent of immobilized chlorine on the samples C is the normality (mol/L) of the titrant sodium thiosulfate solution, V is the volume (L) of the titrant sodium thiosulfate, and W is the weight (g) of the AVM, respectively.

The stability of chlorine on the AVM was evaluated under laboratory conditions (25° C., 15-20% relative humidity) or aqueous solution. For each condition, triplicate membranes with the same size were stored for the predefined time period and titrated for immobilized chlorine content using the above-described iodometric/thiosulfate titration method.

To investigate the chlorine replenishment, the AVM was first chlorinated and titrated as described above. After the first chlorination and titration cycle, the samples were retrieved and washed thoroughly with DI water. This process was defined as one “chlorination-dechlorination” cycle (R1). This process was repeated nine additional times. The process of chlorination and titration was repeated with three parallel samples.

### Material Characterizations

The tensile tests of the membranes were performed using a commercial testing system and analyzed by commercial software. The membranes were cut into a rectangular shape with 50 mm length, 10 mm width, and 2-3 mm thickness. The membranes were stretched until failure at a rate of 5 mm/min. The fiber morphology of the membrane was characterized by scanning electron microscopy (SEM). The pore size of the membranes was measured using an advanced capillary flow porometer with a dry-wet method. Compressed air was used as the flowing gas and Silwick with 20.1 dynes/cm surface tension was used as the wetting liquid to saturate specimens during the wet test. Surface elements of the membranes were analyzed using X-ray Photoelectron Spectroscopy (XPS). The hydrophilic properties of the membranes were characterized with contact-angle goniometer (ramé-hart). The molecular weights of the synthesized polyurethanes were tested by gel permeation chromatography GPC.

### Antimicrobial Efficacy Test

The antibacterial efficacy of AVM was evaluated against several model bacteria: gram-positive S. aureus, gram-negative E. coli, MRSA, VRE, and the army isolated cocktail of 14 strains. Firstly, single colonies of each strain were picked from trypticase soy agar (TSA) plates, resuspended in brain-heart infusion (BHI) medium, and cultured for 16±4 h at 37° C. on a shaking platform at 120 rpm rotation. The cultured bacteria were washed three times with Butterfield's phosphate buffer (BPB) after centrifugation (3000 rpm, 4 min). The bacterial suspensions were then adjusted to ˜5×10⁷ CFU/mL using BPB. Twenty microliters of bacterial suspensions were inoculated on the center of a 1×1 inch sample to achieve an inoculum level of approximately 1×10⁶. After 1, 15, or 30 min of contact, the samples were placed into 10 mL of Na₂S₂O solution (0.02 N) to quench any potential immobilized chlorines in the system, and vortexed for 5 min to detach any residual bacteria. Ten-fold dilution series were performed and 20 μL of every dilution were deposited on TSA plates. Bacteria were incubated at 37° C. for 24 h and viable bacterial colonies were counted and recorded. Each test was repeated three times.

### Bacterial Attachment on the AVM

E. coli was used as a model strain to investigate bacterial attachment onto AVM. A hundred microliter of inoculum containing 10⁷ CFU of E. coli were deposited on AVM samples for 30 min to allow attaching. Then, samples were gently rinsed with excess DI water before staining in Live/Dead Baclight Bacterial Viability Kit per manufacturer's instructions. The stained samples were imaged under an upright microscope. Five images from each sample were randomly captured, and the number of accumulated bacteria on the membranes were counted from captured images.

### Antiviral Efficacy Test

The virucidal efficacy of AVM was tested against three model viruses: enveloped SARS-COV-2, enveloped TGEV as a surrogate for the pathogenic human coronavirus such as SARS-COV-1, and non-enveloped FCV as a surrogate for human norovirus.

ST cells and CRFK cells were maintained in Dulbecco's Modified Eagle's Medium (DMEM) supplemented with 1% penicillin-streptomycin (PenStrep) and 10% heat-inactivated fetal bovine serum (FBS) or horse serum, respectively, and incubated at 37° C. with 5% CO₂. TGEV and FCV were amplified in ST cell and CRFK cell cultures, respectively, to generate the working stocks. SARS-COV-2 strain USA-WA1/2020 was obtained from BEI Resources and propagated to generate a working stock in Vero E6 cells. Vero E6 cells were cultured in complete cell culture media (DMEM supplemented with 5% fetal bovine serum and 1% penicillin-streptomycin). To prepare the test samples, pieces of AVM were cut into 1×1 inch squares with a weight of about 30-34 mg. Melt blown polypropylene fabric (PPN) of N95 respirator was cut into 1×1 inch squares and used as control.

Inactivation of FCV and TGEV on AVM was tested by uniformly depositing 100 μL of viral stock at 10 different points on each sample. After 1-, 10-, 30-, and 120-min of contact, corresponding samples were immersed in 2.0 mL of DMEM medium and vortexed for 1 min to recover remaining virus. After vortex, the sample coupons were removed immediately from the liquid. The chlorine levels in the recovering media were confirmed to be below 0.5 ppm with chlorine testing strips; the recovered virus suspensions were 5-fold serially diluted. Viral infectivity titer before and after contact with samples was assessed by the 50% tissue culture infective dose (TCID₅₀) method. Briefly, corresponding cells of FCV or TGEV were placed in 96-well plates and incubated until 70-80% confluent was reached. Each well of the 96-well plates were washed with PBS and infected with 100 μL of corresponding dilutions with 6 replicates per dilution. The virus solutions were incubated with cell monolayers at 37° C. with 5% CO₂ for 1 h before addition of another 100 μL of cell culture media to reach complete cell media (DMEM supplemented with 1% PenStrep and 10% FBS for TGEV in ST cells, DMEM supplemented with 1% PenStrep and 10% horse serum for FCV in CRFK cells). The plates were incubated for 7 days to allow full cytopathic effects (CPE) to develop. Subsequently, the plates were fixed with 10% paraformaldehyde solution for 1 h and stained with 1% crystal violet solution for 20 min. After a full wash with water, wells of the microtiter plate that were not stained by crystal violet were considered as infected by virus, and wells showing purple stain were considered as not infected by virus. The TCID₅₀ was calculated using the Reed-Muench method (L. J. Reed et al., American Journal of Epidemiology, 27, 493, 1938).

SARS-COV-2 tests were conducted, with all manipulations of SARS-COV-2 done in a biosafety level 3 (BSL-3) laboratory. SARS-COV-2 inactivation on the AVM was examined as follows. Briefly, 100 μL viral stock was uniformly distributed on 10 different points on each sample coupon. Melt-blown polypropylene fabric (PPN) of N95 respirator was cut into 1×1 inch squares and used as a control. After 10-, 30-, or 120-min of contact, sample coupons were immersed in 10.0 mL of complete cell culture media, the same media used to maintain Vero E6 cells (DMEM supplemented with 5% fetal bovine serum and 1% penicillin-streptomycin), and agitated on a platform shaker at 200 rotations per minute for 15 min to recover remaining virus. The extracts were then transferred to a concentrator and centrifuged until the 10 mL starting volume was concentrated to approximately 0.5 mL. Complete cell culture media of Vero E6 cell was added to equilibrate all washed retentates to 2 mL. The sample retentates proceeded to the TCID₅₀ assay similar as above-mentioned protocol for FCV and TGEV, with five replicates per dilution (N=5). The 96-well plates were incubated 72 hours before the determination for CPE on Vero E6 cell monolayers via visual inspection under microscope. Each sample was tested in triplicate (N=3).

For all viral studies, whenever a sample that had less than three out of six wells showing virus positive (for FCV and TGEV) or three out of five wells showing virus positive (for SARS-COV-2) at the lowest dilution factor, the titer was below the limit of detection (LOD) and value of LOD was assigned. The LOD of FCV and TGEV assays are 21 TCID₅₀/sample or 1.32 log (TCID₅₀/sample). The LOD of SARS-COV-2 assay is 26.2 TCID₅₀/sample or 1.42 log (TCID₅₀/sample).

### Cytotoxicity Test

The cytotoxicity of AVM was evaluated against NIH/3T3 Fibroblast, using the MTT cell proliferation assay. NIH/3T3 cells were firstly cultured in DMEM medium supplemented with 10% fetal bovine serum (FBS) and 1% penicillin-streptomycin. About 1×10⁴ cells were seeded into each 96-well TCPS plate for 24 h and then co-cultured with various concentrations (0.1-10 mg/mL) of unchlorinated and chlorinated AVM for 24 h at 37° C. After incubation, 20 μL of 5 mg/mL MTT was added into each well and incubated under 37° C. for 4 h. Supernatant was then removed and 200 μL DMSO was added to dissolve formazan crystals formed. Optical absorbance of the completely dissolved formazan solution was measured at wavelength of 570 nm using an absorbance microplate reader. Each sample was done in six replicates (n=6).

### Water Filter Test

The bactericidal performance of AVM as a water filter was evaluated using a modified water filter method. AVM was firstly cut into circular shape to fit Nalgene analytical filter units. 5×10⁵ CFU of E. coli was then added to 10 mL of DI water and filtered through the analytical filter units under vacuum suction. After filtration, 10 mL of 0.02 N Na₂S₂O₃ solution was added to the bacterial suspension to quench any remaining immobilized chlorine and terminate any bactericidal action. Sterile polystyrene filter membranes with 0.22 μm pore size were used as control. Bacterial dispersion was serially diluted and filtered through the control filter films. The AVM filter membranes were then divided into two triplicate groups: those from one group were put directly onto TSA plates and the rest were vortexed for 3 min to detach residual bacteria. The bacterial suspensions recovered from AVM and filtrates in the lower chamber were serially diluted and inoculated on TSA plates. After the TSA plates were incubated for 24 h at 37° C., viable bacterial colonies were counted and recorded.

### Statistical Analyses

Unless otherwise stated, data are expressed as mean±SEM in the experiments. One-way ANOVA were used to compare different groups of virus titer, followed by Tukey's post hoc test. The level of significance was labeled as NS, *, **, *** and ****, denoting non-significant and p value of <0.05, <0.01, <0.001 and <0.0001. respectively.

### Results

### Design, Fabrication, and Characterization of AVM

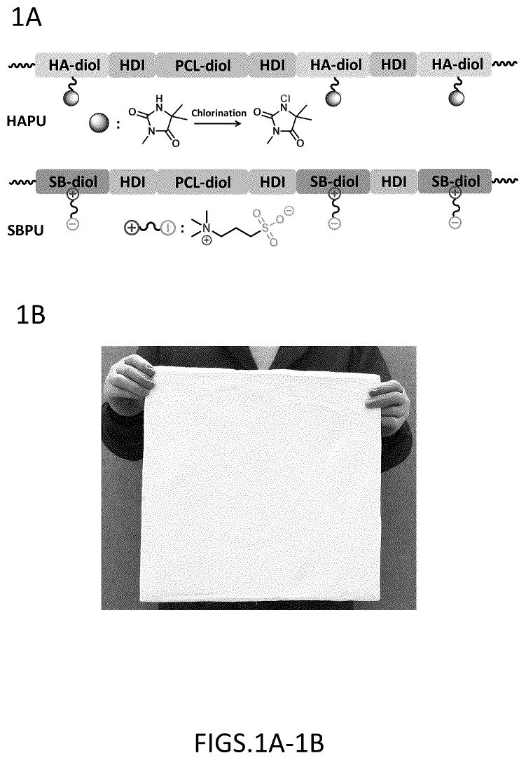

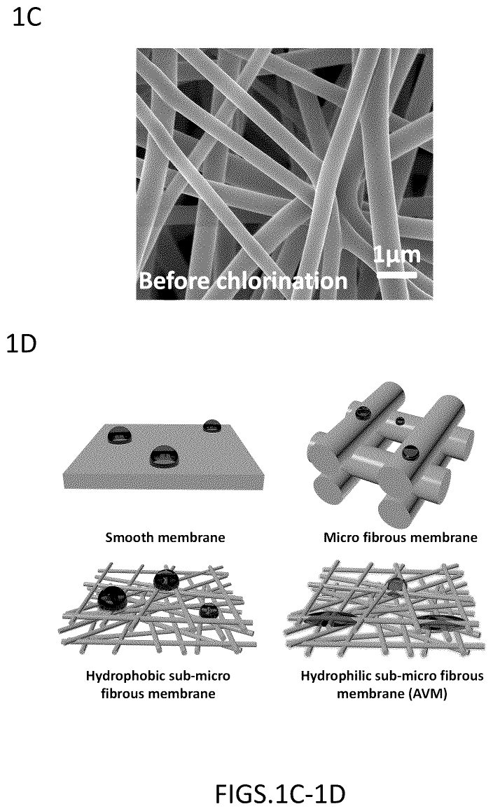

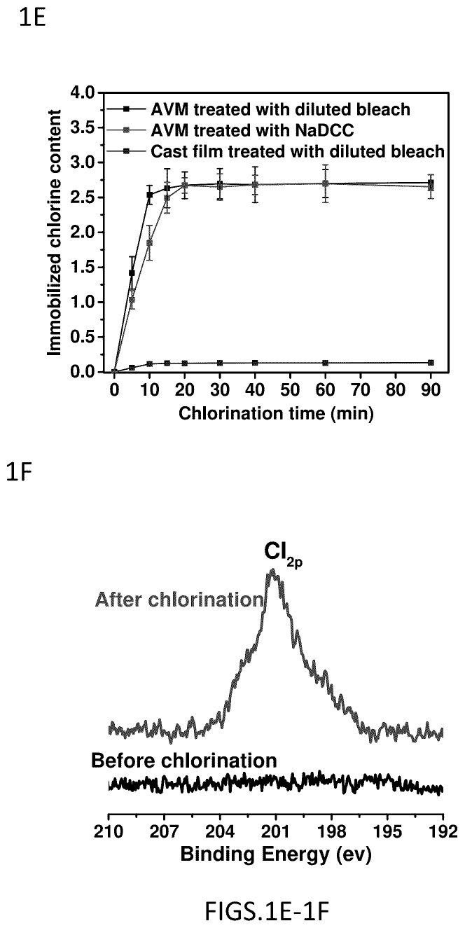

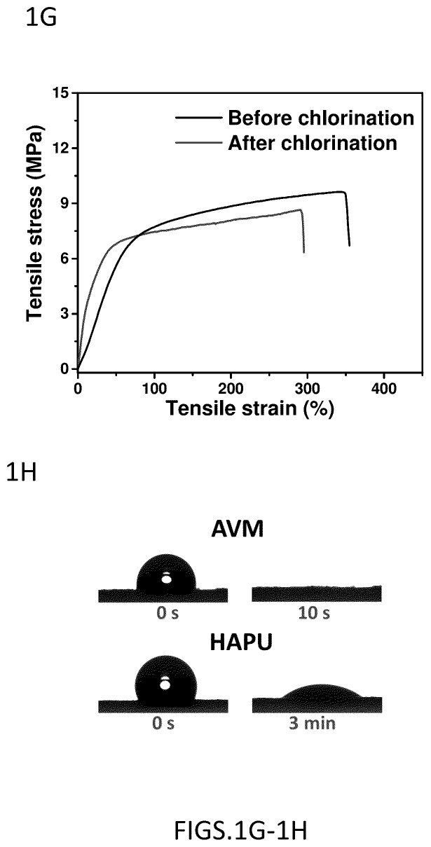

Polyurethanes were selected as the base material, in part, because they are inexpensive, easy to scale, widely used, and tunable to form a wide array of materials from soft elastomers to rigid plastics. To synthesize the polyurethane with the hydantoin group (namely HAPU) and the one with the zwitterionic sulfobetaine group (namely SBPU), HA-diol and SB-diol based monomers, respectively, were first prepared and confirmed by NMR. A polycaprolactone diol (PCL-diol) with a molecular weight of 2 kDa was used as a soft, elastic segment of the polyurethanes, while a 1,6-diisocy anatohexane (HDI) was used as building blocks. The synthesis of HAPU and SBPU (FIG. 1A) was performed according to the following schemes and characterized.

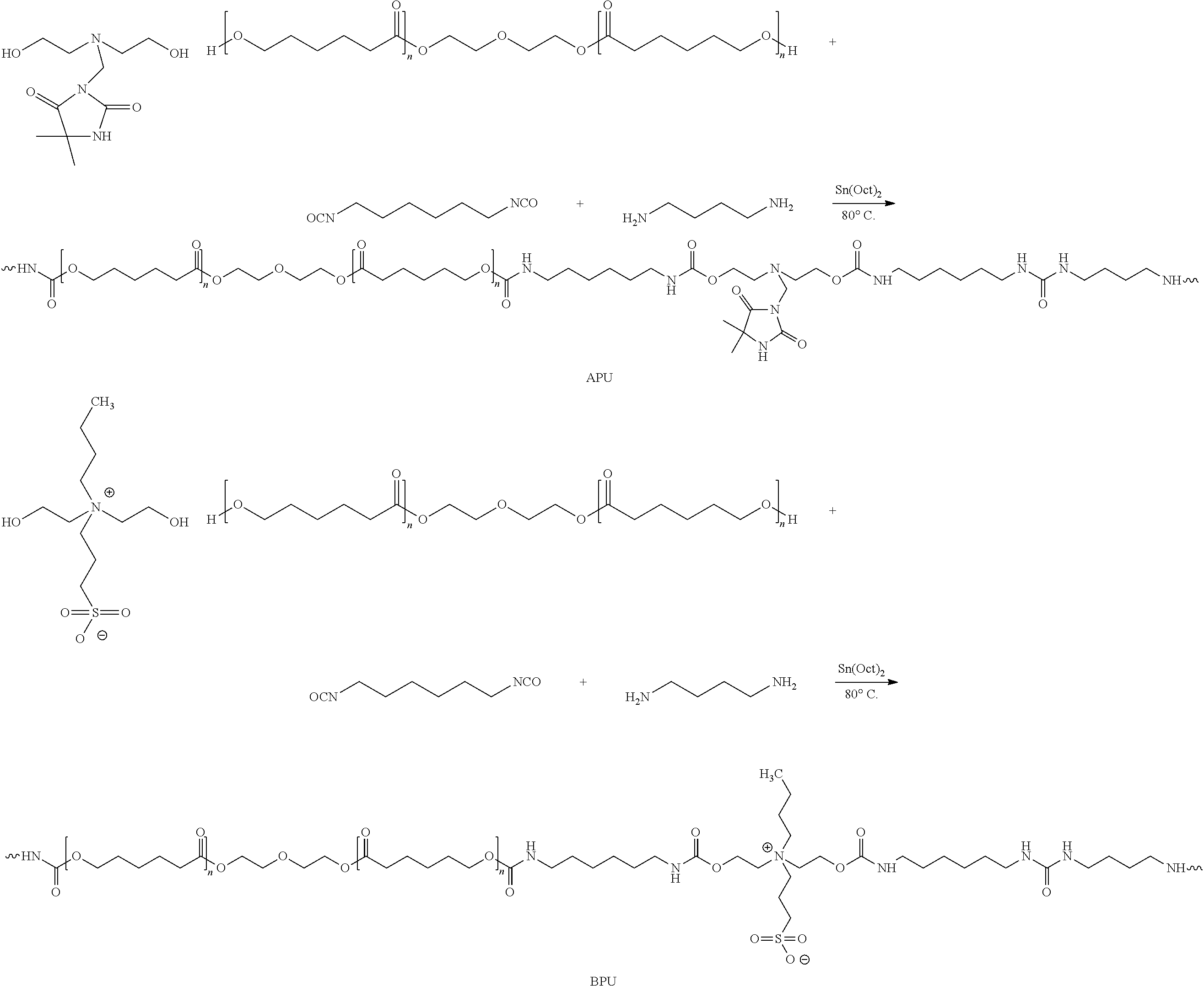

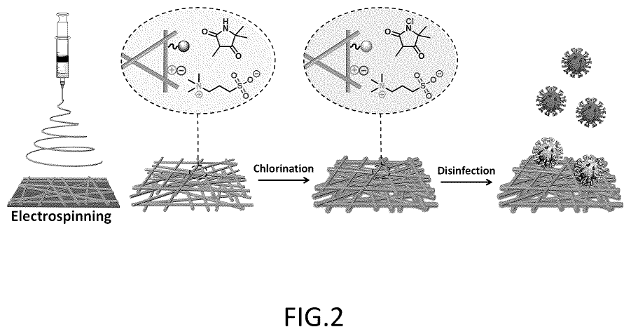

HAPU and SBPU (mass ratio of HAPU and SBPU=3:1) were then blended and the blend was electrospun to fabricate the membrane containing both HA and SB groups (FIGS. 1B, 1C, and 2). The fiber diameter (˜0.6-0.9 μm) and average pore size (˜0.8-2.8 μm) of the membrane was tuned by adjusting the polyurethane concentration. It is important to note that the hydrophilic, sub-micro fibrous structure is highly effective in promoting wetting even when exposed to small size, respiratory droplets (˜submicron to hundreds of microns) which may not wet as readily as smooth membranes, microfibrous membranes, or hydrophobic sub-micro fibrous membranes (FIG. 1D).

The membrane has a high chlorine loading capacity. After treatment with a diluted household bleach or other halogenating agents, such as sodium dichloroisocyanurate (NaDCC) (used for the sterilization of swimming pool and drinking water), the N—H group on the membrane surface was transformed into N—Cl (N-Halamine) structure. The chlorine content, measured via an iodometric titration method, increased with chlorination time (FIG. 1E), saturating at about 2.63% (w/w) after 15 min. The chlorine content was almost 20 times more than that of a cast film made from the same polyurethane blend. The XPS spectrum (FIG. 1F) revealed a peak at 201 eV, attributable to Cl 2p, indicating the formation of N-Halamine (N—Cl) groups after chlorination. In addition, XPS signals at 403 and 168 eV from nitrogen and sulfur, respectively, confirmed the existence of the SB group on the membrane. The chlorinated membrane or AVM was mechanically robust with a tensile strength of ˜8.6 MPa at a strain of ˜295%, slightly lower than before chlorination (FIG. 1G). Moreover, like the untreated membrane, the chlorinated one presented excellent thermal stability. Lastly, the AVM and SBPU membrane were highly wettable with water droplets quickly spreading and becoming imbibed into the membrane within 10 s. In contrast, a water droplet maintained a contact angle after 3 min on the membrane made of HAPU alone (also chlorinated) (FIG. 1H). The fast wetting is important to facilitate intimate contact with viruses inside a respiratory droplet. The large amount of immobilized chlorine and fast wetting make the AVM an ideal candidate to inactivate viruses and bacteria. Furthermore, MTT assays revealed negligible cytotoxicity of the AVM membrane, confirming its safety.

### Anti-Bacterial Properties of AVM

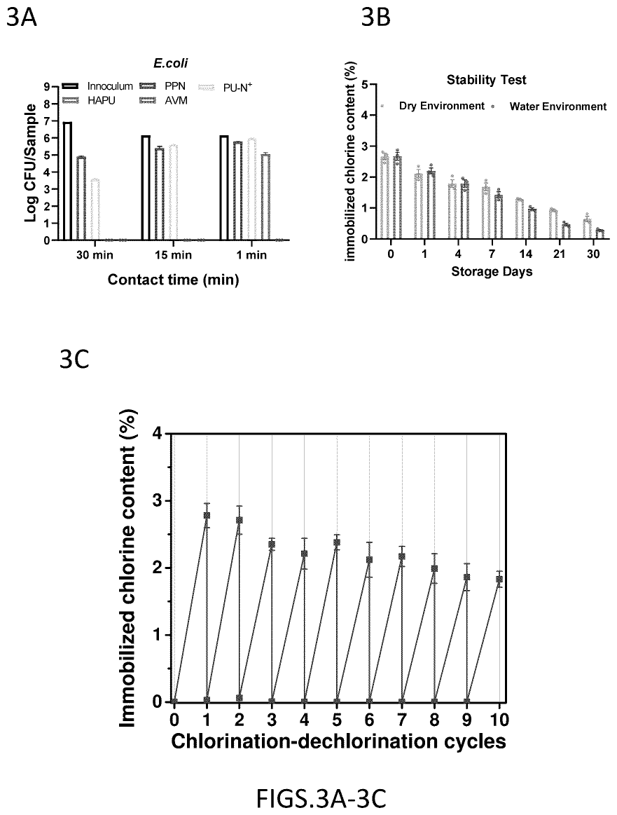

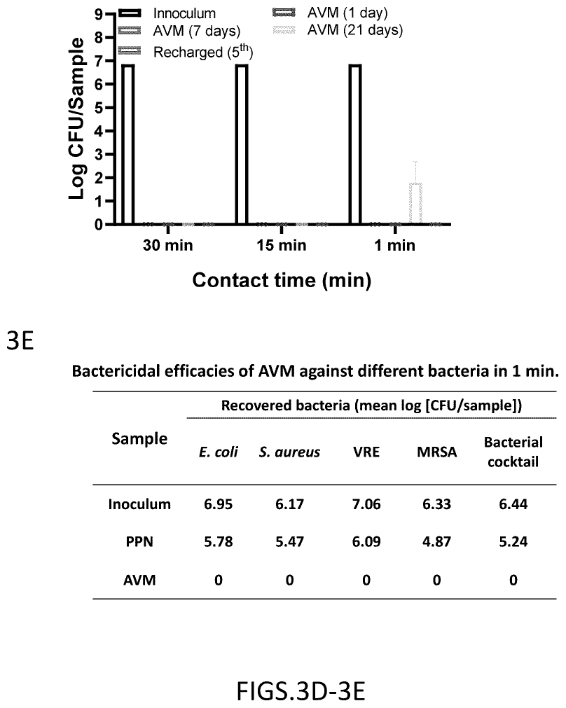

AVMs were prepared and used 7 days after chlorination unless noted otherwise. The bactericidal effect was first evaluated via a direct contact method against five model strains: gram-negative Escherichia coli (E. coli), gram-positive Staphylococcus aureus (S. aureus), vancomycin-resistant Enterococci (VRE), methicillin-resistant S. aureus (MRSA) and a clinically isolated cocktail of 14 strains. In addition, to better assess the bactericidal effect of AVM (chlorinated; 1.68±0.13% chlorine content) 3 controls were included, as follows: a polypropylene non-woven (PPN) membrane (a primary material used in the manufacture of PPE), a sub-micron fibrous membrane made of HAPU alone (also chlorinated; 1.74±0.09% chlorine content), and a sub-micron fibrous membrane made of another type of polyurethane with quaternary amine groups (namely PU-N⁺ membrane) which is known to be effective at inactivating bacteria. About 4.89 log colony forming units per sample (CFU/sample) of E. coli remained on the surface of PPN after 30 min of contact (FIG. 3A). In contrast, AVM and HAPU membranes inactivated all inoculated bacteria, whereas there was 3.61 log CFU/sample of E. coli remaining on the PU-N⁺ membrane. More drastic differences started to emerge as the contact time was decreased. At 15 min, no viable bacteria were detected on AVM or HAPU membranes, but 5.63 log CFU/sample remained on the PU-N⁺ membrane. At 1 min, still no viable bacteria were detected on AVM, a 6-log reduction or a bactericidal efficacy of 99.9999%, whereas neither PU-N⁺ nor HAPU membranes had significant bactericidal effect at this short exposure (FIG. 3A). These results showed superior bactericidal effects of AVM compared to PU-N⁺ or HAPU membranes, likely resulting from the combination of large content of potent chlorine and high wettability of AVM.

Next, the stability and reusability of the AVM in bacteria killing was investigated. The chlorine content of AVM gradually decreased over time under dry condition (25° C., 15-20% relative humidity) and in water, but the decrease was slow and 25% of initial chlorine content remained in dry condition after 30 days (FIG. 3B). Furthermore, the AVMs used in this study were made from one batch of SBPU and four different batches of HAPU. The content and stability of immobilized chlorine on the AVM made from each batch was consistent. Furthermore, the chlorine content of the AVM could be replenished using a household bleach solution for at least 10 chlorination-dechlorination (quenching) cycles (FIG. 3C). The bactericidal effect of AVM was tested 1, 7 and 21 days after chlorination (corresponding to 2.12%, 1.68%, and 0.94% chlorine content, respectively) or after being recharged for the 5th time. Similar biocidal efficacy of 99.9999% for 2 h or 30 min contact time was observed in all cases (FIG. 3D). Only 1.80 log CFU/sample of E. coli was detected on the 21-day old AVM after 1 min exposure, while no bacteria could be detected in any other case. Lastly, when inoculated with S. aureus, VRE, MRSA and the bacterial cocktail, the AVM exhibited similarly robust and ultra-fast biocidal performances (FIG. 3E), achieving more than 6 log CFU reduction within 1 min of contact, in contrast to other antimicrobial membranes which usually required a contact of 30 min to several hours to achieve up to 4 log CFU reduction. All these data showed that the AVM had stable, superior bactericidal effects against a broad spectrum of bacteria for practical uses.

### Anti-Viral Properties of AVM

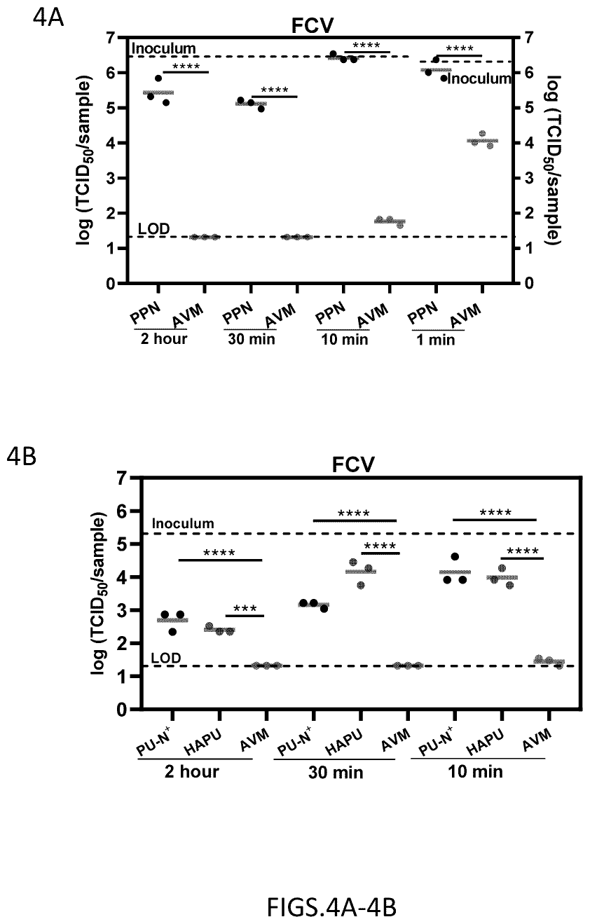

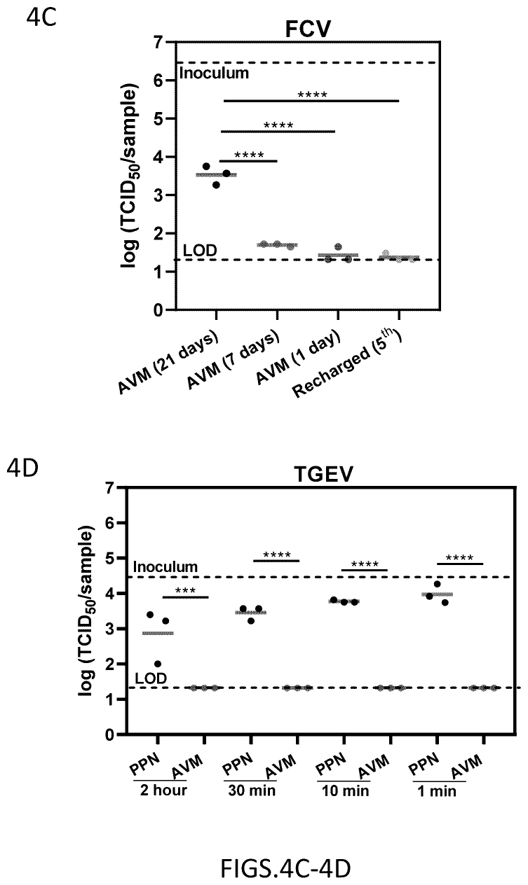

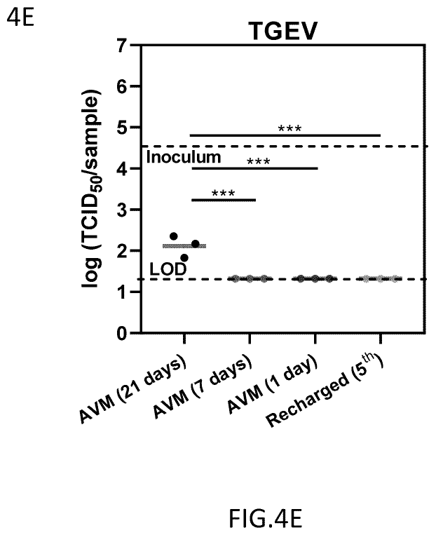

The antiviral activity of AVM against different viruses including SARS-COV-2 was evaluated. A feline calicivirus (FCV) was first chosen since it is a surrogate for human norovirus, which is a leading cause of acute gastroenteritis worldwide. FCV is a non-enveloped, single stranded, positive sense RNA virus Non-enveloped viruses are typically more resistant to disinfectants and other stresses like heat or drying than enveloped ones. The antiviral activity of AVM was analyzed against 100 μL FCV (2.92×10⁶ TCID₅₀/sample) deposited in 10 sperate droplets onto the surface of AVM or PPN (control). Infectious FCV loads remaining on each surface after pre-determined contact periods were quantified on Crandell-Reel feline kidney (CRFK) cells by the 50% tissue culture infective dose (TCID₅₀) method. Cytopathic effects (CPE) were observed under microscope and confirmed after crystal violet staining. While 2.75×10⁵ TCID₅₀/sample of FCV were recovered from PPN membrane after a 2-hour exposure, no CPE was observed either under microscope or under visual inspection in the AVM group, which implies a more than 4.1 log reduction in infectivity in comparison to PPN (FIG. 4A). Similar results were obtained after 30 min of contact. Decreasing contact time to 10 min or 1 min led to almost no virus reduction on PPN membrane, while the AVM reduced the virus titer from 10^{6.47} to 10^{1.75} TCID₅₀/sample in 10 min, or from 10^{6.24} to 10^{4.07} (99.3% inactivation) in just 1 min.

AVM was then compared with HAPU and PU-N′ membranes in FCV inactivation. While the PU-N⁺ membrane had certain antiviral effect, the AVM performed much better with at least 1.37. 1.84 and 2.71 more log reductions after 120-, 30-, and 10-min contact, respectively (FIG. 4B). It may be concluded that N-Halamine based AVM is more potent against FCV than the quaternary amine-based membrane. In comparison with the HAPU membrane with identical chlorine content, the AVM lowered the virus titers by 1.09, 2.84, and 2.54 more logs after 120-, 30-and 10-min contact, respectively. These data confirmed again the importance of the zwitterionic SBPU component that imparted hydrophilicity into the AVM and promoted rapid interactions between the membrane surface and virus, thus leading to enhanced antiviral performance compared to the relatively hydrophobic HAPU membrane. Moreover, the membrane made of HAPU and SBPU without chlorination had substantially no anti-viral effect.

The FCV inactivation was also tested by the AVM after 1, 7 and 21 days of shelf time. No viable FCV was detected on any of the three sets of membranes after 30 min or 2 h contact (FIG. 4C). When the contact time was shortened to 10 min, the FCV titer on 1-day old AVM was reduced from 6.47 to 1.43 log (TCID₅₀/sample); and to 1.70 log (TCID₅₀/sample) or 3.53 log (TCID₅₀/sample) on 7 and 21 day old AVMs, respectively. These results indicate that the 21-day-old AVM had potent antiviral activity after contact as short as 10 min. In addition. AVM after five cycles of quenching/chlorination still maintained robust antiviral behavior (FIG. 4C), which was comparable to the AVM after first chlorination. Even stronger potency was observed against the transmissible gastroenteritis coronavirus (TGEV), an enveloped, positive-sense, single-stranded RNA virus from genus Alphacoronavirus, subgenus Tegacovirus, chosen as a surrogate for other pathogenic human coronavirus such as SARS-COV-1. A lower titer of TGEV (2.8×10⁴ TCID₅₀/sample) was applied to the AVM using the same protocol as for FCV. No viable TGEV was detected on the AVM after as short as 1-min contact (FIG. 4D), while significant infectivity was recovered from the PPN control after all contact times. AVM with various shelf time or recharged all retained the activity against TGEV (FIG. 4E).

### Inactivation of SARS-COV-2

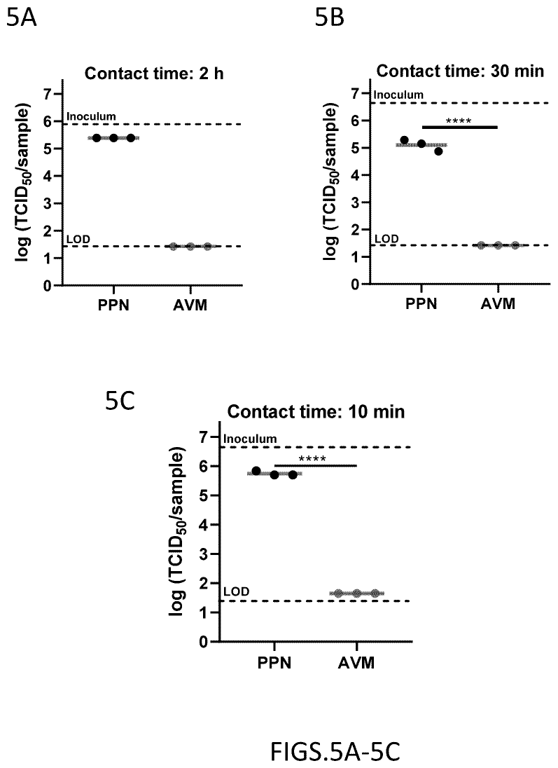

Most significantly, the AVM was similarly effective in inactivating the SARS-CoV-2 that caused this pandemic. AVM was prepared and 7 days later shipped overnight for SARS-COV-2 testing. A total volume of 100 μL inoculum containing 7.8×10⁵ TCID₅₀/sample SARS-COV-2 in 10 separate droplets was applied onto each AVM for 2 h contact. SARS-COV-2 was completely inactivated within the detection limit (LOD) of the assay (1.42 log [TCID₅₀/sample]) (FIG. 5A). In contrast, the virus titer on the PPN membrane control was only slightly reduced from 5.89 to 5.39 log (TCID₅₀/sample). The viral titer on AVM was reduced by 3.97 log and 99.99% in comparison to the PPN membrane.

Next, shorter contact time and higher viral inoculum were tested. A one-hundred microliter bolus containing SARS-COV-2 (6.7×10⁶ TCID₅₀/sample) was inoculated in 10 separate droplets on AVM and allowed to dwell for 30 min. No viable SARS-COV-2 on AVM was detected within the detection limit (FIG. 5B). It yielded more than 3.71 log reduction when compared with the PPN membrane. The contact time was further decreased to 10 min. As expected, a high percentage of virus was recovered from the PPN control (FIG. 5C). In contrast, AVM predictably had an inactivation efficacy at a log reduction of 4.11 compared to PPN. After a mere 10 min, AVM induced viable SARS-CoV-2 titers to decrease from 6.82 log (TCID₅₀/sample) to 1.65 log (TCID₅₀/sample), leading to a 5.17 log reduction or 99.999% of inactivation. These data, together with those from FCV and TGEV, are summarized in Table 1. The data in Table 1 demonstrates the AVM as a superior, broad-spectrum antiviral membrane.

TABLE 1
Virus viability including SARS-CoV-2 on the AVM after different contact time.

| | Inoculum | Contact | | Recovery (TCID₅₀) | Log | | Recovery (TCID₅₀) |
|---|---|---|---|---|---|---|---|
| Virus | (TCID₅₀) | time | | from AVM | reduction | % reduction | from PPN control |
| SARS-CoV-2 | 10^{5.89} | 120 | min | ND | ≥4.47 | ≥99.9966 | 10^{5.39} |
| SARS-CoV-2 | 10^{6.82} | 30 | min | ND | ≥5.40 | ≥99.9996 | 10^{5.13} |
| SARS-CoV-2 | 10^{6.82} | 10 | min | 10^{1.65} | 5.17 | 99.9993 | 10^{5.76} |
| FCV | 10^{6.47} | 120 | min | ND | ≥5.15 | ≥99.9993 | 10^{5.44} |
| FCV | 10^{6.47} | 30 | min | ND | ≥5.15 | ≥99.9993 | 10^{5.11} |
| FCV | 10^{6.47} | 10 | min | 10^{1.75} | 4.72 | 99.9981 | 10^{6.43} |
| FCV | 10^{6.24} | 1 | min | 10^{4.07} | 2.17 | 99.3239 | 10^{6.09} |
| TGEV | 10^{4.45} | 120 | min | ND | ≥3.13 | ≥99.9259 | 10^{2.87} |
| TGEV | 10^{4.45} | 30 | min | ND | ≥3.13 | ≥99.9259 | 10^{3.45} |
| TGEV | 10^{4.45} | 10 | min | ND | ≥3.13 | ≥99.9259 | 10^{3.77} |
| TGEV | 10^{4.45} | 1 | min | ND | ≥3.13 | ≥99.9259 | 10^{3.98} |

Note:
ND indicates not detectable.

### Discussion

There are several design features in the AVM that contribute to its exceptional killing efficacy against bacteria and viruses. In general, there are two main killing mechanisms: contact killing where inactivation occurs after contact, and release killing where the active agent is released into liquid to inactivate bacteria or viruses. The contact killing was likely the primary mechanism for the AVM since no significant free chlorine (less than 0.1 ppm) was detected in media where AVMs were immersed for 10 min, 30 min or 2 h, and no difference in FCV infectivity was observed when the virus was incubated with the leachates or blank culture medium. A diluted commercial bleach at the same chlorine concentration (0.1 ppm) was included as control for the antimicrobial and antiviral tests. The results indicate that such a low chlorine concentration did not have any significant antimicrobial and antiviral properties. Oxidative chlorine atoms from N-halamine can be transferred onto bacterial or viral membranes upon contact and inactivate them with efficacies dependent on the rate and amount of chlorine transfer. An appropriate N-halamine structure with balanced activity and stability is necessary to achieve bacterial or viral inactivation with high and long-lasting efficacy. The chemical stability of N-halamine decreases from amine to amide to imide N-halamine, while the antimicrobial activity increases in the same order (F. Wang et al., Composites Communications, 22, 100487, 2020). Among the amide N-halamines, the one formed by hydantoin group is particularly attractive because of the cyclic structure-enhanced stability and the absence of a-hydrogen (and hence elimination of HCl) In addition, some of the N—H in urethane bonds can be chlorinated and contribute to the overall chlorine content, although the formed N-halamine tends to be less stable. Besides the hydantoin as the main chlorine immobilizing group, the high surface to volume ratio and thus the amount of N-halamine groups on the surface of AVM also contribute to its stable and high chlorine content. Three weeks after preparation, the AVM still contains about 0.94% (w/w) active chlorine and the chlorine can be repeatedly replenished as needed.

In addition to a high immobilized chlorine content, effective contact killing also requires fast and intimate contacts between the surface and the bacteria or viruses. The hydantoin group and the HAPU polymer are relatively hydrophobic. Thus, a zwitterionically modified SBPU was incorporated into the AVM. The sulfobetaine is among the most hydrophilic groups because of its zwitterionic nature. The hydrophilicity combined with the highly porous structure of the sub-micron fibrous surface of AVM resulted in the so-called Wenzel state and facilitated fast and complete wetting. In particular, even small respiratory droplets which may not readily wet conventional surfaces with features larger than the droplets themselves can still be imbibed into the hydrophilic sub-micron fibrous structure due to capillary action. It is also interesting to note that zwitterionic motifs are also known to form a hydration layer on the surface that acts as a physical and energetic barrier to prevent biofouling. The present experiments indeed showed minimal bacteria attached to the surface of SBPU membranes. The amount of dead bacteria attached to the AVM post-killing was on average 20.1% of those attached to the HAPU membranes. It should be noted that unchlorinated SBPU membrane was not able to kill virus or bacteria, although the membrane possessed antifouling properties. The unique N-halamine group, the zwitterionic chemistry, and the sub-micron fibrous structures in the AVM design all played important roles synergistically in achieving the observed antibacterial and antiviral properties.

In the design of the AVM, its practical applications were also considered. For example, the synthesis of the polyurethane base material and the manufacturing of the membrane by electrospinning are both tunable, inexpensive, and scalable. Moreover, the antibacterial activity of the AVM was relatively stable after a single chlorination and under different environmental conditions such as pH and temperature. Since the AVM is also soft, lightweight, breathable, heat-processable, rechargeable and durable, it may be used in a number of applications. It may be attached using adhesive tapes to high-tough surfaces in healthcare facilities or other public spaces such as doorknobs and shopping cart handles to provide continuous protection. It may be incorporated into personal protective equipment using a simple heat press, rendering personal protective equipment (PPE) even safer during wear, reuse or disposal, by adding to it the ability to immediately inactivate infectious pathogens. The AVM may also be used in air and water filters to trap and more importantly kill pathogens. In summary, the AVM is a broad-spectrum, highly effective and reusable antibacterial and antiviral membrane that can inactivates pathogens including SARS-COV-2 within minutes and may be capable to provide long-lasting protections during any local outbreak or global pandemic.

While there have been shown and described what are at present considered the preferred embodiments of the invention, those skilled in the art may make various changes and modifications which remain within the scope of the invention defined by the appended claims.

What is claimed is:

1. A polymeric composition comprising a polyurethane containing at least the following functional groups: (i) a five-membered or six-membered heterocyclic ring containing at least one ring heteroatom selected from nitrogen and oxygen atoms, and (ii) a zwitterionic group.

2. The polymeric composition of claim 1, wherein functional group (i) contains at least two ring heteroatoms selected from nitrogen and oxygen atoms.

3. The polymeric composition according to any one of claims 1 and 2, wherein functional group (i) contains both nitrogen and oxygen ring heteroatoms.

4. The polymeric composition of claim 3, wherein functional group (i) is a hydantoin group or derivative thereof.

5. The polymeric composition according to any one of claims 1-4, wherein the polymeric composition contains a halamine.

6. The polymeric composition according to claim 5, wherein the halamine is present in functional group (i).

7. The polymeric composition according to claim 6, wherein the halamine is a chlorine atom coordinated to a nitrogen atom of functional group (i).

8. The polymeric composition according to any one of claims 1-7, wherein functional group (ii) is a betaine group.

9. The polymeric composition of claim 8, wherein said betaine group is a sulfobetaine group.

10. The polymeric composition according to any one of claims 1-9, wherein the polymeric composition is porous.

11. The polymeric composition according to any one of claims 1-10, wherein the polymeric composition has a fibrous structure.

12. The polymeric composition of claim 11, wherein fibers in the fibrous structure have an average diameter of 0.1-1000 microns.

13. The polymeric composition of claim 11, wherein fibers in the fibrous structure have an average diameter of 0.1-100 microns.

14. The polymeric composition of claim 11, wherein fibers in the fibrous structure have an average diameter of 0.1-10 microns.

15. The polymeric composition according to any one of claims 1-14, wherein the polymeric composition has a membrane structure.

16. The polymeric composition according to any one of claims 1-15, wherein the polyurethane comprises first and second polyurethanes in homogeneous admixture, wherein the first polyurethane contains functional group (i) and not functional group (ii), and the second polyurethane contains functional group (ii) and not functional group (i).

17. The polymeric composition according to any one of claims 1-15, wherein the polyurethane comprises a single polyurethane containing functional groups (i) and (ii).

18. The polymeric composition according to any one of claims 1-17, wherein the polyurethane further comprises at least one of polyester and polyether linkages.

19. The polymeric composition according to any one of claims 1-18, wherein the polyurethane further comprises urea linkages.

20. An article comprising the polymeric composition according to any one of claims 1-19.

21. The article of claim 20, wherein the article is a door knob, handle, counter, video screen, tool, filtration membrane, or protective suit.

22. A method for inactivating microbes and viruses on a surface of an object, the method comprising incorporating an anti-microbial anti-viral polymeric composition in a surface of said object, wherein said polymeric composition comprises a polyurethane containing at least the following functional groups: (i) a five-membered or six-membered heterocyclic ring containing at least one ring heteroatom selected from nitrogen and oxygen atoms, and (ii) a zwitterionic group.

23. The method of claim 22, wherein functional group (i) contains at least two ring heteroatoms selected from nitrogen and oxygen atoms.

24. The method according to any one of claims 22 and 23, wherein functional group (i) contains both nitrogen and oxygen ring heteroatoms.

25. The method of claim 24, wherein functional group (i) is a hydantoin group or a derivative thereof.

26. The method according to any one of claims 22-25, wherein the polymeric composition contains a halamine.

27. The method according to claim 26, wherein the halamine is present in functional group (i).

28. The method according to claim 27, wherein the halamine is a chlorine atom coordinated to a nitrogen atom of functional group (i).

29. The method according to any one of claims 22-28, wherein functional group (ii) is a betaine group.

30. The method of claim 29, wherein said betaine group is a sulfobetaine group.

31. The method according to any one of claims 22-30, wherein the polymeric composition is porous.

32. The method according to any one of claims 22-31, wherein the polymeric composition has a fibrous structure.

33. The method of claim 32, wherein fibers in the fibrous structure have an average diameter of 0.1-10 microns.

34. The method according to any one of claims 22-33, wherein the polymeric composition has a membrane structure.

35. The method according to any one of claims 22-34, wherein the polyurethane comprises first and second polyurethanes in homogeneous admixture, wherein the first polyurethane contains functional group (i) and not functional group (ii), and the second polyurethane contains functional group (ii) and not functional group (i).

36. The method according to any one of claims 22-34, wherein the polyurethane comprises a single polyurethane containing functional groups (i) and (ii).

37. The method according to any one of claims 22-36, wherein the polyurethane further comprises at least one of polyester and polyether linkages.

38. The method according to any one of claims 22-37, wherein the polyurethane further comprises urea linkages.

39. The method according to any one of claims 22-38, wherein the object is an article designed for contact with a bodily part.

40. The method according to any one of claims 22-38, wherein the object is designed to inactivate a microbe or virus in a liquid or gaseous sample.

41. The method according to any one of claims 39 and 40, wherein the article is a door knob, handle, counter, video screen, tool, filtration membrane, or protective suit.

42. The method according to any one of claims 22-41, wherein the method inactivates a microbe.

43. The method of claim 42, wherein the microbe is selected from bacteria, archaebacteria, protists, and fungi.

44. The method according to any one of claims 22-41, wherein the method inactivates a virus.

45. The method of claim 44, wherein the virus is an enveloped or non-enveloped virus.

46. The method of claim 44, wherein the virus is a coronavirus.

47. The method according to any one of claims 22-46, wherein the object is constructed of the polymeric composition.

48. The method according to any one of claims 22-46, wherein the object is coated with a film of the polymeric composition.
